(19) United States
(12) Patent Application Publication
(10) Pub. No.: US 2012/0303284 A1
(43) Pub. Date: Nov. 29, 2012

(54) DETERMINATION OF TIMING OF CHEMOTHERAPY DELIVERY

(75) Inventors: Alexey A. Leontovich, Rochester, MN (US); Svetomir N. Markovic, Rochester, MN (US); Leonid V. Ivanov, Grinnell, IA (US)

(73) Assignee: MAYO FOUNDATION FOR MEDICAL EDUCATION AND RESEARCH, Rochester, MN (US)

(21) Appl. No.: 13/515,915

(22) PCT Filed: Nov. 1, 2010
(86) PCT No.: PCT/US10/54976

§ 371 Date: Aug. 9, 2012

Related U.S. Application Data
(60) Provisional application No. 61/287,495, filed on Dec. 17, 2009.

(51) Int. Cl. G01N 33/48; G06F 19/00

(52) U.S. Cl. 702/19

(57) ABSTRACT

A system and method for determination of one or more favorable time(s) for chemotherapy or other pharmacological treatment delivery analyze time-dependent fluctuations of at least one biological variable measured in blood samples obtained from clinical patients and determine one or more favorable times for the pharmacological treatment of the patient. In some examples, the biological variables are immune variables.

### TECHNICAL FIELD

The disclosure relates to planning of chemotherapy treatment.

### BACKGROUND

Over the last several years, there has been an increasing understanding that the reasons for the unrealized potential of cancer immunotherapeutics may lay in the state of the immune system in patients with cancer. Most solid tumors contain many non-malignant cells which make up the inflammatory tumor microenvironment. These cells express an immunosuppressive phenotype and act to support cancer growth, invasion, and metastasis, while effectively “shielding” the tumor from the surrounding immune system. An illustrative example are tumor infiltrating regulatory T-cells (Treg) that have been shown to significantly suppress tumor-specific immune responses, thereby promoting rather than suppressing cancer development.

The relationship of cancer and immunity (inflammation) has yielded a number of efforts to correlate measured variables of inflammation with clinical outcomes in patients with advanced malignancies. Measurement of plasma concentration of inflammatory, “acute-phase reaction”, proteins (e.g. C-reactive protein) has been investigated as both a risk factor and a prognostic variable in various human malignancies. Elevated serum levels of several acute phase reactants has been shown to be associated with risk of recurrence, tumor burden, disease progression, presence of anorexia-cachexia syndrome and decreased overall survival in many cancers. The most extensively studied acute phase reactant is C-reactive protein (CRP). Since its discovery in 1930, CRP has been extensively used as a sensitive, albeit nonspecific biomarker of inflammation. In humans, plasma CRP is a positive acute-phase protein, the levels of which rise more than 100-fold in the setting on an inflammatory stimulus. This reflects increased synthesis of CRP, mainly in hepatocytes, induced by pro-inflammatory cytokines such as interleukin-6 (IL-6). After the onset of an acute inflammatory stimulus, CRP can be detected in plasma within 4 to 6 hours with a peak at around 48 hours. CRP half-life is approximately 19 hours and it is fairly constant; therefore the main determinant of the circulating plasma levels is the production rate. Once the inflammation resolves, the CRP plasma level quickly return to normal; unless it is kept elevated by continued production in response to ongoing inflammation and/or tissue damage. Thus, the “acute phase response” is a dynamic process of “up” and “down” regulation of the immune system that fluctuates over time.

### SUMMARY

In general, the disclosure relates to planning delivery of chemotherapy treatment. In general the systems and/or methods described herein utilize concentration measurements of at least one biological variable to judge the level of systemic inflammation in patients with metastatic melanoma. The systems and/or methods analyze time-dependent fluctuations of at least one biological variable measured in blood samples obtained from clinical patients and determine one or more favorable times for the pharmacological treatment of the patient.

In one example, a method comprises receiving time series data of immune variable concentration for an observed time period for each of a plurality of identified immune variables, fitting a periodic function to the time series data corresponding to each of the plurality of identified immune variables, calculating a treatment prediction parameter based on a relative concentration and a relative differential of the fitted periodic function for each time series data that fits a periodic function, choosing the proposed treatment date such that the treatment prediction parameter is maximized, and reporting the proposed date of treatment that maximizes the treatment prediction parameter.

In another example, a computer-readable medium is encoded with instructions that cause a programmable processor to receive time series data of immune variable concentration for an observed time period for each of a plurality of identified immune variables, fit a periodic function to the time series data corresponding to each of the plurality of identified immune variables, define a relative concentration of the fitted periodic function based on a maximum immune variable concentration within the observed time period, a minimum immune variable concentration within the observed time period, and an extrapolated immune variable concentration on a proposed treatment date, define a relative derivative of the fitted periodic function based on a maximum derivative within the observed time period, a minimum derivative within the same period, and an extrapolated derivative on the proposed treatment date, calculate a treatment prediction parameter based on the relative concentration and the relative differential, choose the proposed treatment date such that the treatment prediction parameter is maximized, and report the proposed date of treatment that maximizes the treatment prediction parameter.

In another example, a system comprises a controller that receives a set of time series data of immune variable concentration for an observed time period for each of a plurality of identified immune variables, a curve-fitting module executed by the controller that fits a periodic function to the set of time series data corresponding to each of the plurality of identified immune variables, a treatment prediction parameter module executed by the controller that calculates a treatment prediction parameter based on a relative concentration and a relative differential of the periodic function for each set of time series data that fits a periodic function, a proposed treatment date module executed by the controller that chooses the proposed treatment date such that the treatment prediction parameter is maximized, and a reporting module executed by the controller that generates a report concerning the proposed date of treatment that maximizes the treatment prediction parameter.

In another example, a method comprises receiving a plurality of time series data of immune variable concentration in a patient for an observed time period each corresponding to a different one a plurality of identified immune variables, determining for which of the identified immune variables the corresponding time series of immune variable concentration fit a periodic function, for those immune variables that fit a periodic function, defining a relative concentration of the fitted periodic function based on a maximum immune variable concentration within the observed time period, a minimum immune variable concentration within the observed time period, and an extrapolated immune variable concentration on a proposed treatment date, for those immune variables that fit a periodic function, defining a relative derivative of the fitted periodic function based on a maximum derivative within the observed time period, a minimum derivative within the same period, and an extrapolated derivative on the proposed treatment date, calculating a treatment prediction parameter based on the relative concentration and the relative differential, choosing the proposed treatment date such that the treatment prediction parameter is maximized, and reporting the proposed date of treatment for the patient that maximizes the treatment prediction parameter.

In another example, a method of determining one or more favorable treatment times for delivery of pharmacological treatment to a patient comprises receiving a set of time series data for each of one or more biological variables, determining whether each set of time series data fit a periodic function, for each biological variable that fits a periodic function, computing a treatment prediction parameter, and determining one or more favorable treatment times based on the treatment prediction parameter calculated for at least one of the biological variables that fits a periodic function.

The details of one or more examples are set forth in the accompanying drawings and the description below. Other features and/or advantages will be apparent from the description and drawings, and from the claims.

### BRIEF DESCRIPTION OF DRAWINGS

FIGS. 1A-1C are flowcharts illustrating an example overall process for determination of time(s) for delivery of chemotherapy treatment.

FIGS. 2A and 2B show the frequency of 9 example functions as concentration dynamics of 28 cytokines and 25 cell subtypes for 10 patients.

FIG. 3 shows the sum of ranks for each of the 10 patients compared with the clinical outcome for each individual patient.

FIG. 4 shows extrapolated relative CRP concentration (right axis, dashed bars) and relative first derivative of the fitted function on the day of treatment (left axis, black bars) as related to PFS of the patients.

FIG. 5 shows the relationship between progression free survival (PFS) time (days) and sum of ranks of IL-12p70 and CD197/CD206 ratio.

FIGS. 6 and 7 show nonlinear regression fitting of CD197/CD206 ratio time dependent fluctuations in patients #1 (PFS=916 days) and patient #2 (PFS=37 days).

FIGS. 8A-8C show synthetic virtual concentration/cell count curves showing dynamic of one variable in several patients.

FIGS. 9A and 9B show relative concentration (right axis, dashed bars) and relative first derivative of the fitted function on the day of treatment (left axis, black bars) as related to PFS of the patients.

FIG. 10 is a block diagram illustrating an example system for determination of time(s) for delivery of chemotherapy treatment.

FIG. 11 illustrates an example simulation which considered three different observation periods (10, 15 and 20 days), three various sampling frequency (every day, every other day and 1-2 days), one hundred amplitudes and twenty periods

FIGS. 12A-12C are graphs illustrating example frequency distribution of R² for various ranges and datasets.

FIGS. 13A-13C are graphs illustrating example frequency distribution of R² for an example 5-2-5 sample collection schedule.

FIG. 14 is a graph illustrating example frequency distribution of R² for an example 5-2-5 sample collection schedule.

FIG. 15 is a chart illustrating an example association between the 5-day period of actual chemotherapy application, time predicted by the example clustering algorithm and PFS in 8 melanoma patients.

FIGS. 16A-16C are example graphs illustrating counts of variables profiles for IL-12p70 (FIG. 16A), IL-17 (FIG. 16B) and CRP (FIG. 16C).

FIGS. 17A and 17B are example graphs illustrating example clustering of concentration profiles IL-1ra and IL-12p70 in Patient #1 (PFS=916 days) (FIG. 17A) and concentration profiles IL-1ra and IL-12p70 in Patient #2 (PFS=37 days) (FIG. 17B).

### DETAILED DESCRIPTION

In general, the example systems and/or methods described herein analyze time-dependent fluctuations of at least one biological variable measured in blood samples obtained from clinical patients and determine one or more relatively more favorable times for the pharmacological treatment of the patient. The systems and/or methods determine relatively more favorable time(s) for chemotherapy delivery based on serial measurements of the one or more biological variables. In some examples, the biological variables are immune variables. The determination may be patient-specific in the sense that only those biological variables satisfying desired threshold values may be used to determine favorable treatment times for each individual patient.

The measurements of the one or more biological variables may be indicative of the level of systemic inflammation in cancer patients. In the examples described herein, the techniques are described with respect to patients with metastatic melanoma. However, the techniques may also be applied to patients with other types of cancer.

To identify which of the biological variables are indicative of favorable time(s) to deliver treatment to these patients, the systems and/or methods ascertain whether or not one or more biological variables are stable or variable over time, and if variable, in what systemic immune context. That is, curve-fitting is applied to time series data for each patient to determine the best fit variable function for each of the measured biological variables.

Once the best fit variable function is established, the treatment planning techniques described herein therapeutically utilize the variation of one or more biological variables over time information and devise a treatment strategy which, by using timed administration of conventional cytotoxic therapy (chemotherapy), may augment anti-tumor immunity and affect clinical outcomes.

In an example clinical trial described herein, the patient population included patients with unresectable stage IV malignant melanoma. Eligible patients had unresectable, histologically confirmed stage IV disease, age over 18 years, measurable disease as defined by the Response Evaluation Criteria in Solid Tumors (RECIST), Eastern Cooperative Oncology Group (ECOG) performance status (PS) of 0-2, and life expectancy ≧3 months. Both newly diagnosed, previously untreated patients, as well as patients who have had prior therapy for their metastatic disease were enrolled.

Treatment was initiated with temozolomide (TMZ) 150 mg/m² on days 1-5 on cycle 1 and the dose was increased to 200 mg/m² for all subsequent cycles if tolerated. Patients were treated every 4 weeks until progression, unacceptable toxicity or patient refusal. Prior to initiation of first chemotherapy cycle, eligible patients underwent peripheral blood testing for immunological biomarkers (immune variables) every 2-3 days for a period of two weeks. The blood samples were tested for a total of 52 variables; that is, 52 measurements of cytokine concentrations and cell counts in blood samples. The 52 variables are listed in Table 1.

TABLE 1

| | Variable |
|---|---|
| 1 | IL-10 |
| 2 | IL-12p70 |
| 3 | G-CSF |
| 4 | IL-9 |
| 5 | VEGF |
| 6 | CD206 |
| 7 | IL-1ra |
| 8 | IL-13 |
| 9 | CD4/294 |
| 10 | CD11c/14 |
| 11 | CD197/CD206 |
| 12 | DR(hi) |
| 13 | IL-15 |
| 14 | IL-17 |
| 15 | IL-6 |
| 16 | IL-8 |
| 17 | Eotaxin |
| 18 | TGF-b (ng/ml) |
| 19 | CD11c/CD123 |
| 20 | Treg (% gated) |
| 21 | IL-4 |
| 22 | IL-5 |
| 23 | GM-CSF |
| 24 | MIP-1a |
| 25 | MIP-1b |
| 26 | CD3−/16+56 |
| 27 | CD3−/CD16− |
| 28 | TIM3:CD294 |
| 29 | DR/11c (DC1) |
| 30 | DR/123 (DC2) |
| 31 | B7-H1(DRhi) |
| 32 | IL-7 |
| 33 | FGF |
| 34 | IFN-g |
| 35 | IP-10 |
| 36 | CD3/4 |
| 37 | CD3/8 |
| 38 | CD4/TIM3 |
| 39 | B7-H1(DRlo) |
| 40 | Treg (% total) |
| 41 | CRP pmol/L |
| 42 | IL-1b |
| 43 | IL-2 |
| 44 | RANTES |
| 45 | TNF-a |
| 46 | CD3/62L |
| 47 | CD197 |
| 48 | MCP-1 |
| 49 | PDGF |
| 50 | CD3 |
| 51 | DR(lo) |
| 52 | CD3/69 |

The time series of six CRP concentration measurements was fitted to a sine curve. The curve was then extrapolated for two periods and the next consecutive peaks of CRP concentration were predicted. Based on the periodicity of CRP oscillations, TMZ chemotherapy was initiated prior to the estimated time of the next CPR peak, or on day 14 post-registration if the peak could not be identified.

Peripheral blood samples were obtained at baseline and every 2-3 days thereafter for 15 days prior to the first cycle of TMZ chemotherapy. In order to study the global behavior of the anti-tumor immune response, the samples were further analyzed for plasma concentration of 29 different cytokines/chemokines/growth factors and the percentage of 22 immune cell subsets. All biospecimens were collected, processed, and stored in uniform fashion following established standard operating procedures. To reduce inter-assay variability, all assays were batch-analyzed after study completion.

The data was obtained as follows. However, it shall be understood that the data could be obtained in other ways, and that the disclosure is not limited in this respect. Peripheral blood mononuclear cell (PBMC) immunophenotyping for immune cell subset analysis. Blood was separated into plasma and PBMC using a density gradient (Ficol-hypaque, Amersham, Uppsala, Sweden). Plasma samples were stored at −70° C., and PBMC were stored in liquid nitrogen. PBMC bio-specimens were analyzed for the frequencies of T cells (CD3+), T helper cells (CD3+4+), CTL (CD3+8+), natural killer cells (NK, CD16+56+), T helper 1 (Th1) cells (CD4+TIM3+), Th2 cells (CD4+294+), T regulatory cells (Treg, CD4+25+FoxP3+), type 1 dendritic cells (DC1, CD11c+HLA-DR+), DC2 (CD123+HLA-DR+), type 1 macrophages (M1, CD14+197+), type 2 macrophages (M2, CD14+206+) and for the activation status of these cell types. Immunophenotyping of PBMC was performed by flow cytometry using FITC- and PE-conjugated antibodies to CD3, CD4, CD8, CD16, CD56, CD62L, CD69, TIM3, CD294, HLA-DR, CD11c, CD123, CD14, CD197, CD206, and B7-H1 (Becton-Dickinson, Franklin Lakes, N.J.). In addition, intracellular staining for FoxP3 (BioLegend, San Diego, Calif.) was performed according to the manufacturer's published instructions. Data were processed using Cellquest® software (Becton-Dickinson, Franklin Lakes, N.J.). In order to access the Th1/Th2 balance PBMC were stained with anti-human CD4, CD294, and TIM-3. The stained cells were analyzed on the LSRII (Becton Dickinson Franklin Lakes, N.J.). The CD4 positive population was gated and the percent of CD4 cells positive for either CD294 or TIM-3 was determined Preliminary data suggests that CD4/CD294 positive Th2 cells exclusively produce IL-4 and not IFN-γ upon PMA and ionomycin stimulation. Conversely, CD4/TIM-3 positive Th1 cells exclusively produce IFN-γ and not IL-4 following the same in vitro stimulation. Enumeration of Treg was performed using intracellular staining for FoxP3 of CD4/25 positive lymphocytes.

Protein levels for 29 cytokines, chemokines, and growth factors, including IL-1β, IL-1rα, IL-2, IL-4, IL-5, IL-6, IL-7, IL-8, IL-9, IL-10, IL-12(p70), IL-13, IL-15, IL-17, basic fibroblast growth factor (FGF), Eotaxin, granulocyte colony-stimulating factor (G-CSF), granulocyte-macrophage colony-stimulating factor (GM-CSF), interferon γ IFN-γ), 10 kDa interferon-gamma-induced protein (IP-10), macrophage chemoattractant protein 1 (MCP-1), migration inhibitory protein 1α (MIP-1α), MIP-1β, platelet-derived growth factor (PDGF), Regulated upon Activation Normal T-cell Expressed and Secreted (RANTES), tumor necrosis factor α (TNF-a), vascular endothelial growth factor (VEGF), CRP, and transforming growth factor beta (TGF-β1) were measured using the BioRad human 27-plex cytokine panel (Cat #171-A11127, Bio-Rad, San Diego Calif.) as per the manufacturer's instructions. Plasma levels of TGF-β1 were determined using the duoset capture and detection antibodies (R and D Systems Minneapolis, Minn.) as per manufacturer's instructions. Briefly, plasma samples were treated with 2.5 N Acetic acid and 10M urea to activate latent TGF-β1 followed by neutralization with NaOH and HEPES. The activated samples were added to plates, which had been coated with a mouse anti-human TGF-β1. After incubation the wells were washed and biotinylated chicken anti-human TGF-β1 detection antibody was added. The color was developed using streptavidin-HRP and R and D systems substrate kit. Plasma levels of TGF-β1 were calculated using a standard curve from 0-2000 pg/ml.

All plasma cytokine measurements were performed in duplicate. Normal values for plasma cytokine concentrations were generated by analyzing 30 plasma samples from healthy donors (blood donors at the Mayo Clinic Dept. of Transfusion Medicine). A set of three normal plasma samples (standards) were run along side all batches of plasma analysis in this study. If the cytokine concentrations of the “standard” samples differed by more than 20%, results were rejected and the plasma samples re-analyzed.

The data for each of the variables was then applied to a curve fitting process to determine whether each cyctokine concentration/cell count followed a predictable variation over time. For example, the data for each variable was applied to each of the functions shown in Table 2:

TABLE 2

| | |
|---|---|
| F1 | Linear function y = ax + b |
| F2 | Exponential Fit: y = ae\hat{ }(bx) |
| F3 | Exponential Association: y = a(1 − exp(−bx) |
| F4 | Logistic Model: y = a/(1 + b * exp(−cx)) |
| F5 | Quadratic Fit: y = a + bx + cx\hat{ }2 |
| F6 | Sinusoidal Fit: y = a + b * cos(cx + d) |
| F7 | Rational Function: y = (a + bx)/(1 + cx + dx\hat{ }2) |
| F8 | Gaussian Model: y = a * exp((−(b − x)\hat{ }2)/(2 * c\hat{ }2)) |
| F9 | MMF Model: y = a * b + c * x\hat{ }d)/(b + x\hat{ }d) |
| F0 | No Fit |
| F0 | No DATA |

In the example described herein, CurveExpert 1.4 software (Daniel G. Hyams Hixson, Tenn.) and GraphPad Prizm 4.0 software (GraphPad Software Inc. La Jolla Calif.) were used to construct time-dependent profiles of plasma cytokine concentrations and immune cell counts by fitting data points to the selected mathematical functions. Both software packages use Levenberg-Marquart (LM) algorithm to solve nonlinear regressions to fit experimental data to a model curve. The correlation coefficient r=√(Sₜ−Sᵣ)/Sₜ calculated by CurveExpert may be used as the first criterion for goodness of fit, where Sₜ considers the distribution around a constant line and is calculated as Sₜ=Σ(y−yᵢ)² and Sᵣ considers the deviation from the fitting curve and is calculated as Sᵣ=Σ(yᵢ−f(xᵢ))². GraphPad Prizm was used to obtain R² values, 95% confidence intervals for the variables of the fitted functions, and 95% confidence bands for the fitted curves. R² is calculated as R²=1−Sᵣ/Sₜ. These parameters may be used as selection criteria in different steps of the analysis as described below.

Although specific commercially available software packages are described herein to perform the curve fitting analysis, it shall be understood that other software packages or custom software could also be used to perform the curve fitting analysis, and that the disclosure is not limited in this respect. In addition, mathematical methods other than an Levenberg-Marquart (LM) analysis, such as Fourier transform, autocorrelation methods, or other mathematical of determining or identifying a periodic pattern in a data set, may be used can be used to reveal periodical pattern of concentration and cell count fluctuation and define the function.

The purpose of the curve fitting analysis is to determine whether any of the measured immune variables change in a predictable fashion following a cyclical pattern (dynamic equilibrium of immunity and cancer). Therefore, the goal of the curve fitting analysis is to assess whether concentrations of plasma cytokines/chemokines and immune cells fluctuate, and if so, to determine whether these fluctuations follow a mathematically predictable cyclical pattern. To that end, the plasma levels for the 52 immune variables (29 different cytokines/chemokines/growth factors and the percentage of 22 immune cell subsets) in serial blood samples collected every 2-3 days prior to initiation of TMZ therapy were measured in 10 patients with metastatic malignant melanoma. Of the 12 enrolled patients, number of data points was inadequate for curve-fitting analysis in two patients; one patient was hospitalized shortly after enrollment, and the other had an insufficient number of successive blood samples obtained prior to initiation of TMZ therapy. Technical reproducibility was assessed by the coefficient of variation among duplicates (average coefficient of variation was 5.13% for 1593 data points).

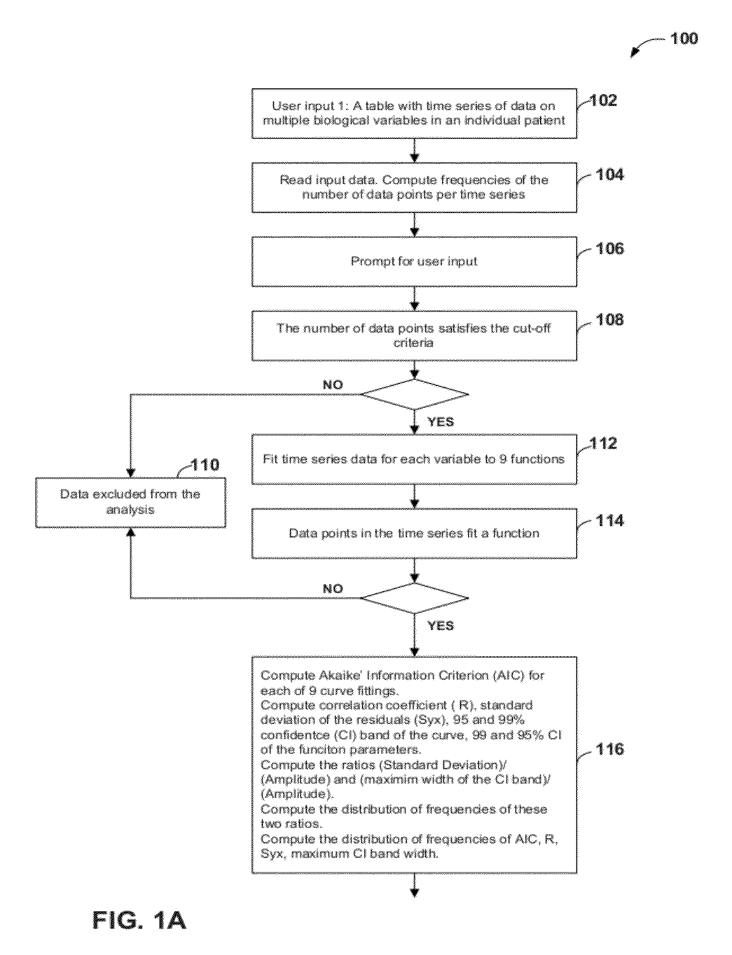

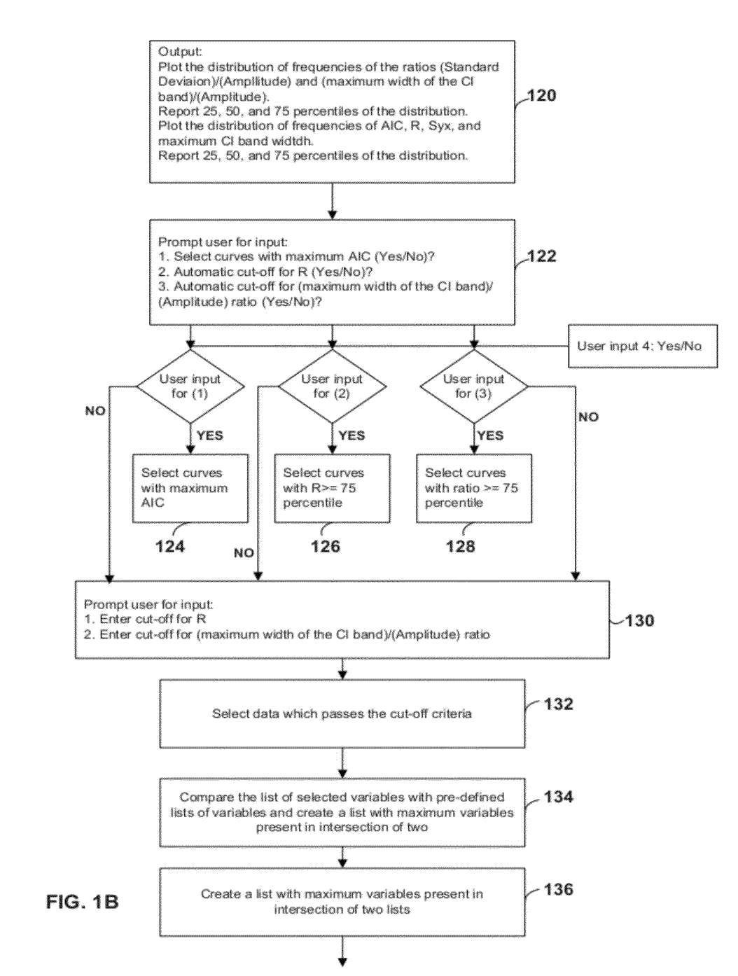

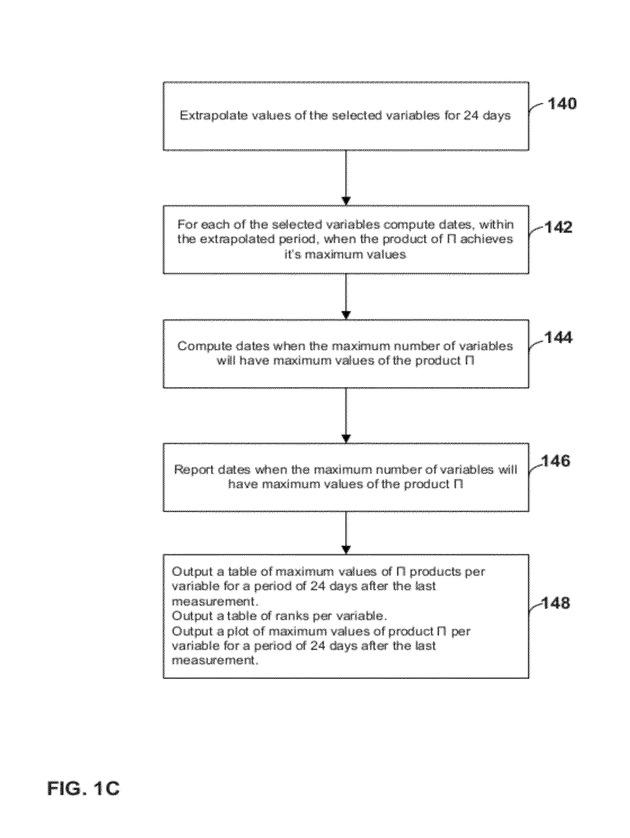

FIGS. 1A-1C are flowcharts illustrating an example overall process 100 for determination of favorable times for delivery of chemotherapy or other pharmacological treatment. For purposes of the present description, cytokine concentration or cell counts will be denoted as “immune variables” and cytokine concentration or cell count measured in an individual patient on a specific day as a data point. Time-dependent profiles for each variable and each patient were constructed by fitting the data points to each of 10 possible functions (e.g., the 9 mathematical functions plus “no fit” function listed in Table 2).

FIG. 1A shows the process by which presence of a regular pattern in fluctuation of cytokines' concentration and cell counts is determined FIG. 1B shows the process of determining the correlation between clinical outcome and the presence of a pattern in the variance of the immune variables. FIG. 1C shows an example process by which a proposed time of therapy for a particular patient may be determined based on the curve fitting(s) for one or more selected immune variables.

The curve fitting analysis was performed based on 6 or 7 sequential measurements (time points) for each variable/patient over a period of 15-days. The “goodness of fit” of the measured variables with a mathematically predicted function was estimated statistically using the correlation coefficient calculated by CurveExpert 1.4 software (REF/source). The cut-off criteria for good fit were computed as follows: (a) the frequency distribution of the correlation coefficient was computed across all profiles and all patients; and (b) the value of the 75^{th} percentile (0.86) was accepted as a cut-off to eliminate profiles which did not fit a model well.

As shown in FIG. 1A, the process receives time series of data on one or more biological immune variables in an individual patient (102) and a date of treatment start. To ensure that each time series includes sufficient data to perform each curve fitting, the process computes the frequencies of the number of data points per time series (104). If the number of data points does not satisfy a user input cut-off criteria, the data may be excluded from the analysis.

If the number of data points satisfies the user input cut-off criteria (106, 108), the process fits the time series data for each immune variable to each of a set of mathematical functions (112). In this example, the process fits the time series data to each of the 9 functions listed in Table 2. However, it shall be understood that more or fewer functions may be used, and that other functions not listed in Table 2 may also be used, and that the disclosure is not limited in this respect.

If the data points fit a function (114), the process may compute various parameters indicative of the “goodness” of the fit of the time series data to each of the functions (116). For example, the process may compute Akaike's Information Criterion (AIC) for each of 9 curve fittings; compute a correlation coefficient (R), a standard deviation of the residuals (S_{yx}), 95 and 99% confidence (CI) band of the curve, 99 and 95% CI of the function parameters; compute the ratios (Standard Deviation)/(Amplitude) and (maximum width of the CI band)/(Amplitude); compute the distribution of frequencies of these two ratios; and/or compute the distribution of frequencies of AIC, R, S_{yx}, maximum CI band width.

As shown in FIG. 1B, the process may next report and/or plot the distribution of frequencies of the ratios (Standard Deviation)/(Amplitude) and (maximum width of the CI band)/(Amplitude); report 25, 50 and 75 percentiles of the distribution; plot the distribution of frequencies of AIC, R, S_{yx}, and maximum CI band width; report 25, 50 and 75 percentiles of the distribution (120). It shall be understood that more or fewer of these parameters may be computed and/or plotted, and that other parameters not specifically shown herein may be determined, and that the disclosure is not limited in this respect.

The process may next prompt user for input (122). For example, the process may prompt the user to input one or more of the following: 1. Select curves with maximum AIC (Yes/No)? (124); 2. Automatic cut-off for R (Yes/No)? (126); 3. Automatic cut-off for (maximum width of the CI band)/(Amplitude) ratio (Yes/No)? (128).

If the user does not enter automatic cut-offs, the process may prompt user for input 913). For example, the process may prompt the user to: 1. Enter cut-off for R; and/or 2. Enter cut-off for (maximum width of the CI band)/(Amplitude) ratio.

The process may then select the immune variables corresponding to the data series which pass the cut-off criteria (132). The process may then compare the list of selected immune variables with lists of pre-defined variables (determined by, for example, the ranked list of immune variables) (134). The process may then find an intersection set of the two lists which contains the maximum number of immune variables (136). The process may then create a list of these immune variables and continue the analysis with this list.

The resulting list contains those immune variables having the highest correlation with PFS for that particular patient.

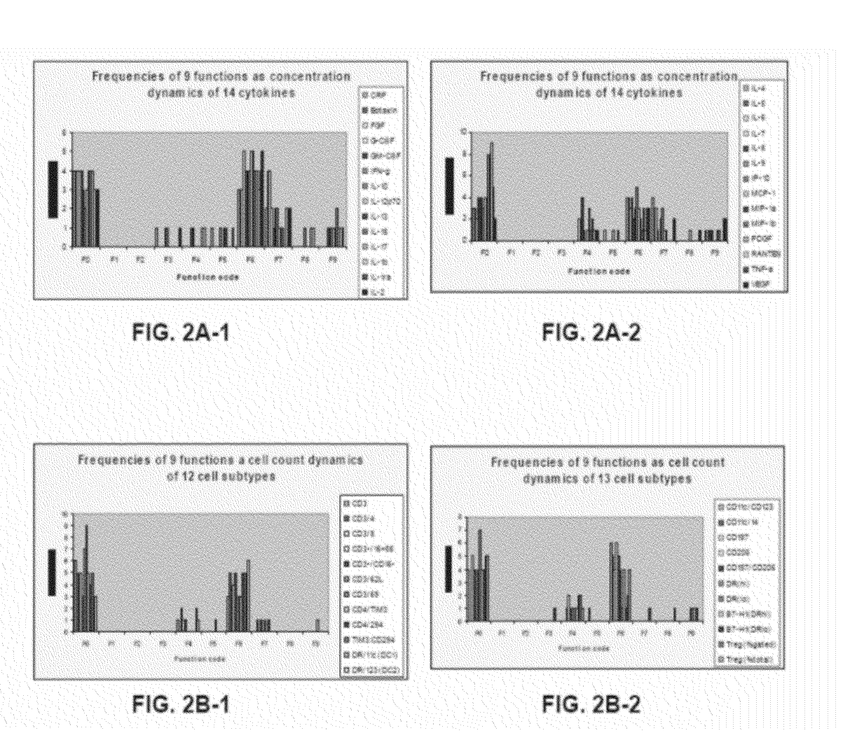

FIGS. 2A-1 and 2A-2 show the frequency of the 9 example functions as concentration dynamics of 14 cytokines and 14 cytokines, respectively, for 10 patients. FIG. 2B-1 and 2B-2 show the frequency of the 9 functions as cell count dynamics of 12 cell subtypes and 13 cell subtypes, respectively, for 10 patients. The cytokine legend and color code is described on the right-side of figure. Function codes: F1=Linear function y=ax+b; F2=Exponential Fit: y=aê(bx); F3=Exponential Association: y=a(1−exp(−bx); F4=Logistic Model: y=a/(1+b*exp(−cx)); F5=Quadratic Fit: y=a+bx+cx̂2; F6=Sinusoidal Fit: y=a+b*cos(cx+d); F7=Rational Function: y=(a+bx)/(1+cx+dx̂2); F9=Gaussian Model: y=a*exp((−(b−x)̂2)/(2*ĉ2)); F10=mMF Model: y=(a*b+c*x̂d)/(b+x̂d); F0=No Fit/No data.

The example distributions shown in FIGS. 2A and 2B frequencies of all 9 mathematical models (functions) shows that most time-dependent profiles fit sinusoidal or rational functions.

In order to establish whether an ordered pattern of fluctuation correlates with clinical outcome (progression free survival or PFS), an index of fitness is assigned to each variable, patients are ranked by the sum of indices, and the correlation coefficient between this rank and the PFS is calculated. In one example, the assigned index was 1 if the profile fitted a function well (correlation coefficient ≧0.86) and the function was biologically possible. Functions with infinite growth or infinite decline were considered biologically implausible as their extrapolation produces biologically impossible values (e.g. <0) for plasma cytokine concentrations or cell count frequencies and were assigned an index of zero (0). The index was −1 if a profile did not fit any function. Using these criteria, the sum of these indices was then calculated for each immune variable per individual patient.

For example, if IL-10 concentration dynamically fitted to cosine, rational or logistic functions in 7 patients and fitted an exponential growth (biologically impossible) function in one patient, this would produce a score of 7 (7×1+0=7). Table 3 shows the rank for each of the 52 immune variables in the example clinical trial.

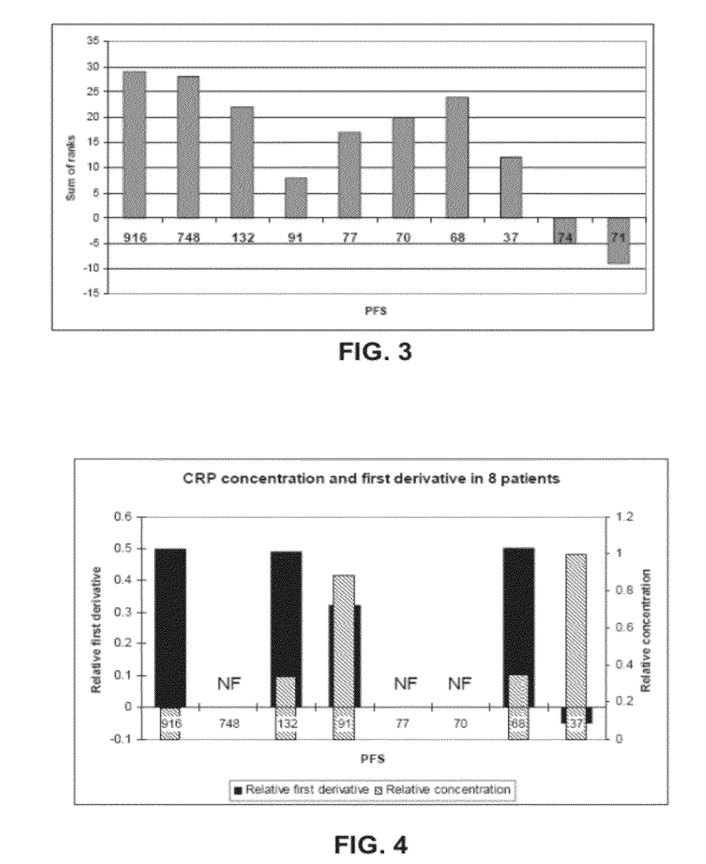

FIG. 3 shows the sum of ranks for each of the 10 patients compared with the clinical outcome for each individual patient. The data suggests that the patients with the highest rank (fluctuation of cytokine concentrations and/or cell counts follows an ordered pattern) experienced the best clinical outcomes (PFS of 916 and days for ranks 29 and 28, respectively). Surprisingly, the subjects with the lowest (−5 and −9, respectively) rank score (entirely random fluctuation of cytokine concentrations/cell counts) identified by this method were the two patients with metastatic ocular melanoma. These two patients were not studied further given the inability to fit them to any mathematical model.

Separate analysis of the remaining eight patients with metastatic cutaneous melanoma resulted in a correlation coefficient between the total individual score and PFS of 0.72. In a similar way scores (sum or indices) were assigned to each variable. In this case indices were summed across patients per individual variable. Table 3 shows the resulting rank for each of the 52 example immune variables.

TABLE 3

| Rank | Variable |
|---|---|
| 7 | IL-10, IL-12p(70), G-CSF |
| 6 | IL-9, VEGF, CD206 |
| 5 | IL-1rα, IL-13, IL-15, IL-17, CD4/294, CD11c/14, CD197/CD206, |
| | DR(hi) |
| 4 | IL-6, IL-8, Eotaxin, TGF-b, Treg (% gated) CD11c/CD123 |
| 3 | IL-4, IL-5, GM-CSF, MIP-1a, MIP-1b, CD3−/16+56+, |
| | CD3−/CD16−, DR/11c (D1), DR/123(D2), TIM3:CD294, |
| | B7-H1(DRhi) |
| 2 | IL-7, FGF, IFN-g, IP-10, CD3/4, CD3/8, CD4/TIM3, |
| | B7-H1(DRlo), Treg (% total) |
| 1 | CRP, IL-1b, IL-2, RANTES, TNF-α, CD3/62L, CD197 |
| 0 | MCP-1, PDGF, CD3, DR(lo) |
| −1 | CD3/69 |

Determining which immune variables correlate with clinical outcome. In order to understand if certain of the measured immune variables of immune function had a greater/lesser impact on survival, as measured by cyclical function, additional analyses were performed on the 14 variables assigned a score of 5 or greater in the 8 patients with metastatic cutaneous melanoma (see, e.g., Table 3).

As described above, the index assigned to each variable was 1 if the profile fits a function, 0 for time dependent profiles of variables which fitted biologically impossible functions, and −1 if a profile did not fit any function. As the maximum theoretical score of an immune variable was 8 in this example (8 patients), the cut-off of 5 was chosen because it eliminated those variables which fit a function in <50% of patients. In the case of larger trials (more patients) the cutoff could be chosen appropriately. The maximum score obtained for the remaining variables was 7. These included IL-1rα, IL-9, IL-10, IL-12(p70), IL-13, IL-15, IL-17, G-CSF, VEGF, Th2 T-helper lymphocyte subset (CD4/294), CD11c-positive monocytes (CD11c/14), the ratio of polarized M1/M2 macrophages (DD197/CD206) and DR(hi).

FIG. 1C illustrates an example process by which further analysis was performed on eight patients on variables with the score 5 or greater. The plasma cytokine concentration or the cell count was extrapolated on the day of treatment for the 14 selected variables in the eight patients analyzed (140). The first derivative of the fitted function on the day of treatment was calculated. The first derivative shows whether the function at that point is increasing (positive value), decreasing (negative value) or is not changing (zero) and the magnitude of the first derivative reflects the magnitude of the trend.

The range of plasma cytokine concentrations/cell counts varied significantly across patients. In order to be able to compare these concentrations in different patients, the concentrations/cell counts may be convereted into relative values by using the formula:

relative conc(“conc”)=(Cₘₐₓ−Cₑₓ)/(Cₘₐₓ−Cₘᵢₙ), where
Cₘₐₓ is the maximum concentration within the observed time period,
Cₘᵢₙ is the minimum concentration within the same period, and
Cₑₓ is the extrapolated concentration on the day of treatment.

The same conversion was applied to first derivative values. In the cases when both maximum and minimum first derivative were negative the following formula may be applied:

relative derivative(“der”)=−1*(1−(Dₘₐₓ−Dₑₓ)/(Dₘₐₓ−Dₘᵢₙ)), where
Dₘₐₓ is the maximum derivative within the observed time period,
Dₘᵢₙ is the minimum derivative within the same period, and
Dₑₓ is the derivative of the function for the extrapolated point corresponding to the day of treatment in order to compensate for the subtraction of two negative numbers.

The initial hypothesis was that application of treatment near the CRP concentration peak may be therapeutically advantageous by predicting the correct time point in the cycle when chemotherapy will selectively deplete replicating Tregs and other immunosuppressive elements and “unblock” the anti-tumor immune response. However, final data analysis showed no correlation between PFS and CRP concentration or the first derivative of the fitted function (see, e.g., FIG. 4) (correlation coefficients −0.47 and −0.36 respectively).

In this example, a single parameter may be used to characterize both the magnitude of change and the trend of the fluctuation for a given biological variable. This parameter may then be used to find a relationship between the fluctuation of plasma cytokines/immune cellular elements and clinical outcome and guide personalized “timed” chemotherapy delivery. In some examples, this parameter (referred to as index Pi or Π) may be obtained by exponentiating the relative concentration and the first derivative and calculating their product with the formula:

Π=e^{der×T}×e^{conc}, where
e^{der} is the number e (2.7182818 . . . ) raised to the power of the relative derivative,
e^{conc} is the number e raised to the power of relative concentration, and
T is function period in days to correct for variable period length.

The index Pi, as a product based on both the relative concentration and the relative derivative, takes into consideration both the magnitude of the concentration and the dynamic trend of a given variable at a precise time point in the immune response cycle, hence describing the time-dependent fluctuation of a certain immune biomarker more accurately than the protein concentration or cell count alone.

In the above example, index Pi is a product of exponentiated values, therefore it is converted into a sum by the transformation: e^{der×T}×e^{conc}=e^{der×T)+conc}. Generally in these examples the parameters of interest are der and conc, and the exponent alone may be taken as follows:

Π=(der×T)+conc, where
der is the relative derivative,
conc is the relative concentration, and
T is function period in days to correct for variable period length.

In other examples, a parameter Π could be computed that does not include the period (T). For example, T could be left out of either of the above equations, or out of other appropriate equations. Other equations may also be used. In general, an equation may include the value and magnitude of the trend without zeroing the product (unless both values are actually zero—which makes a zero legitimate result). That is, an index Pi may be calculated by any formula or numerical transformation which produces a linear or non-linear dependency of the result on both arguments (relative derivative and relative concentration) with the limitation that the result is zero only when both arguments equal zero. If one of the arguments equals zero, the equation does not produce a zero result.

In another example, relative values of the concentration and derivative can be calculated from the maximum and minimum values of the curve, fitted to the experimental data points.

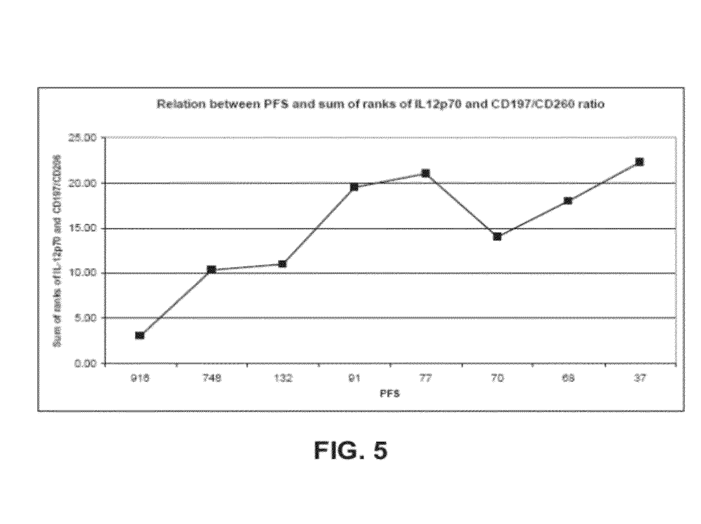

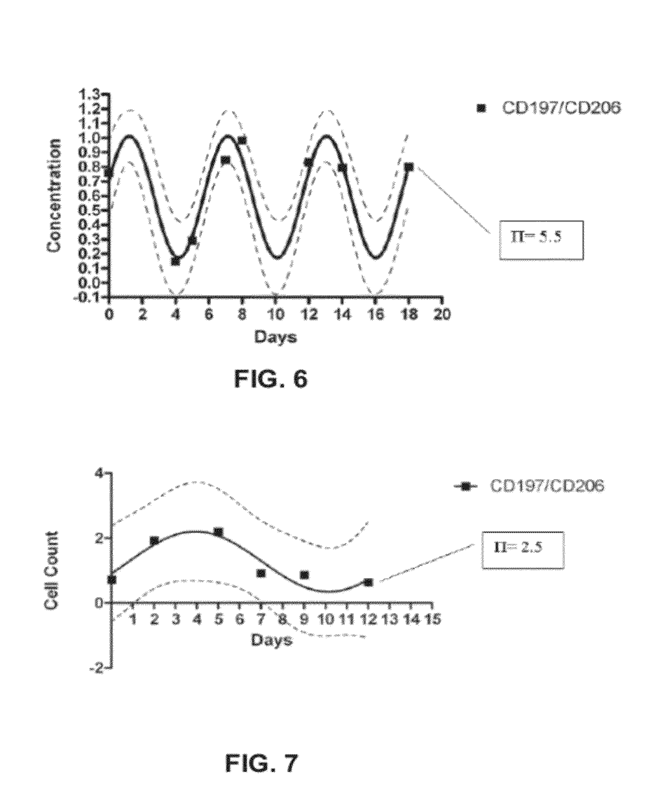

In terms of clinical application the aim was to determine a relationship between concentration and dynamic trend of the variable at the day of treatment with clinical outcome. With this in mind, the goal was to find the variables with the highest correlation between the product Π on the day of treatment and PFS. In order to do that, the products Π were ranked in descending order for each measured immune variable. If an immune variable did not fit a biologically possible function, then the product could not be calculated and since 14 immune variables were analyzed and the lowest rank for a product was 14, it follows was the next lowest rank for a product which could not be calculated was 15. Because this rank is weighted by the proportion of non-fitted variables in a given patient, a weighted rank was used calculated as 15*(number of immune variables which do not fit a function)/(total number of measured variables). In this example, the correlation coefficient was used to assess the association between the rank of each of these 14 variables and the patients' PFS. In this example, two immune variables, the concentration of IL12p70 and the ratio of CD197/CD206 positive cells (ratio of polarized M1/M2 macrophages) had the highest correlation coefficients of −0.73 and −0.62, respectively. This was further supported by a correlation coefficient of −0.83 between the sum of the ranks for these two variables and PFS. Four patients (50%) with the sum of ranks of these two variables below 15 had average PFS of 466, whereas the other four with sum of ranks above 15 had average PFS of 68 (see, e.g., FIG. 5), suggesting that the value of the product Π on the day of treatment correlated favorably with clinical outcome. For instance, the product Π on the day of treatment for the patients at the two extremes were 5.5 in the patient with the highest PFS (916 days; corresponding rank=1) and 2.5 in the subject with the lowest PFS (37 days; corresponding rank=10) (see, e.g., FIGS. 6 and 7) Therefore, application of treatment at a time point when this product is elevated, meaning that the concentration is high and also on the rise, results in improved outcome.

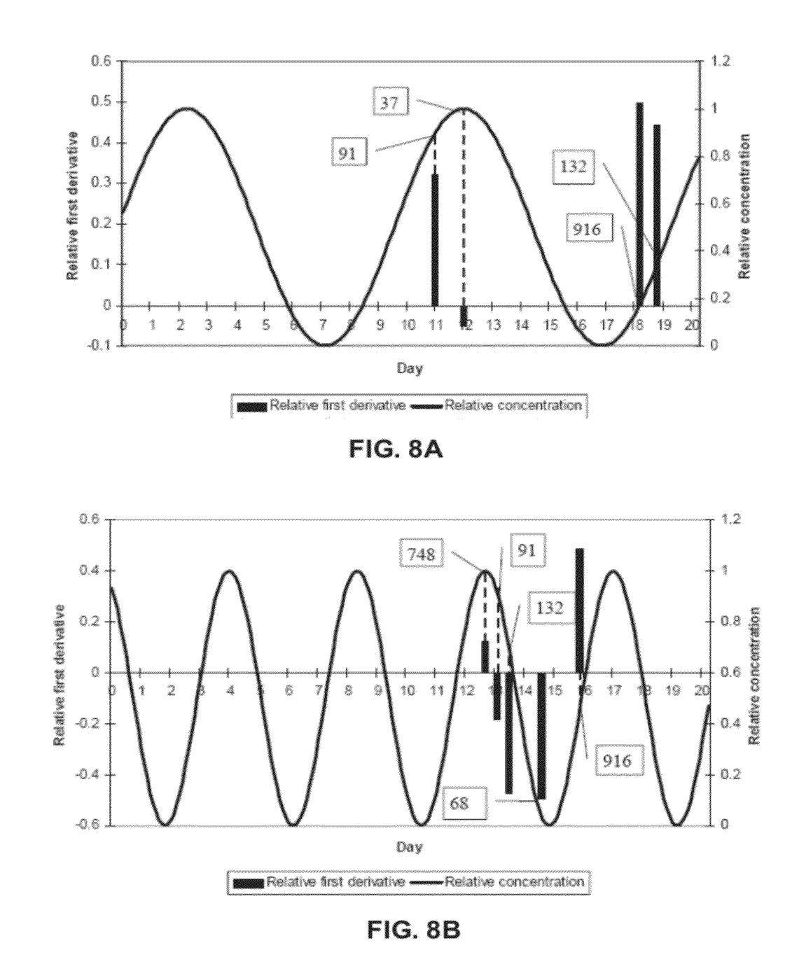

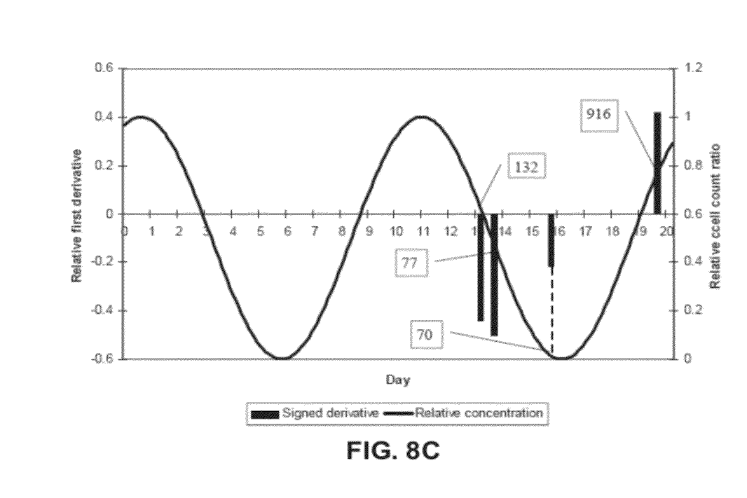

To better understand how the concentration of a cytokine or cell count and the trend for increase or decrease of these variables (first derivative of the fitted function) are related to the clinical outcome, the values of these variables in patients with different PFS were compared. A fitted cosine curve was computed where all four parameters of the cosine function (a, b, c and d) were average values of the corresponding parameter across patients being compared and a variable being analyzed. The resulting curve represented averaged concentration/cell count dynamics for several patients on a relative concentration scale (calculation of relative concentration is described above). First derivatives of the fitted function on the treatment day were also plotted on a relative scale (FIGS. 8A-8C). In effect, the plot shows relative concentration and relative first derivative on the treatment day for several patients with different PFS. Concentration/cell count and first derivative plots were constructed for CRP, IL-12p70 and CD197/CD206 for patients in whom these variables fitted a cosine function. These figures demonstrate, that the clinical outcome (PFS) directly correlated with concentration or first derivative for the given measurements (FIGS. 8A-8C).

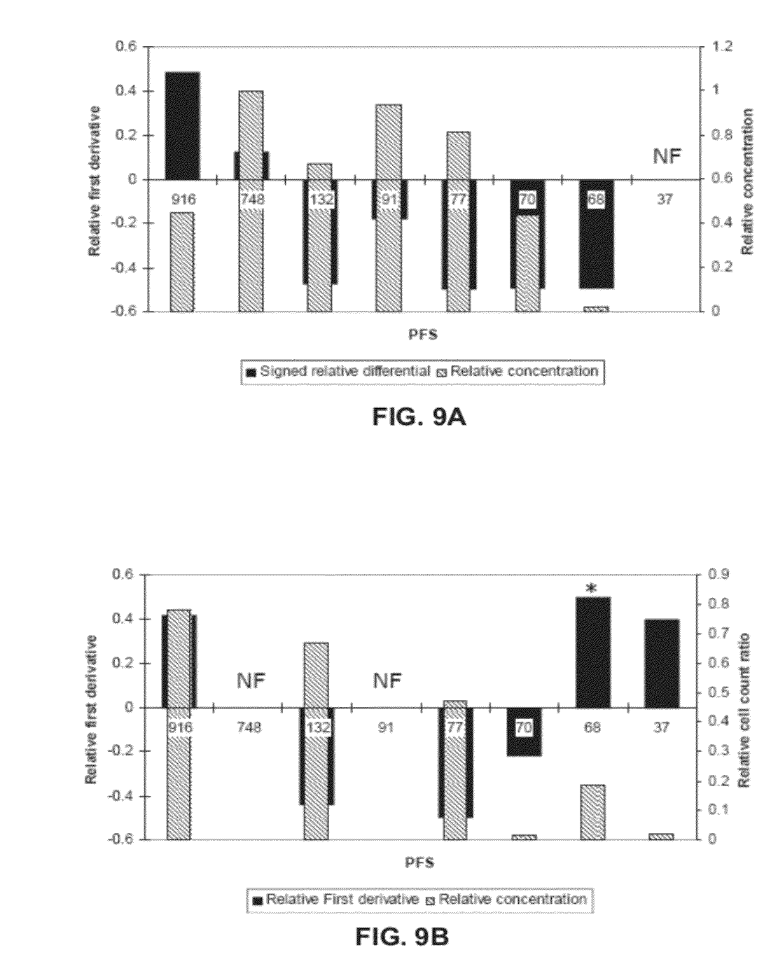

In attempt to further generalize this observation, concentration/cell count ratio and first derivative of on the day of treatment across 8 patients for IL-12p70, CRP and CD197/CD206 were compared. The values were compared as relative values for a given variable in each patient. FIGS. 9A and 9B demonstrate improved clinical outcome in those patients in whom the treatment was applied at a concentration peak or strong increase trend of IL-12p70 and CD197/CD206.

Patterns of periodicity of sinusoidally fluctuating immune variables. Since a large proportion of time dependent profiles were fitted to cosine curves when a rather non-stringent criterion (the correlation coefficient) was used, only those data which fitted cosine curves with the value of R2 greater than the 75 percentile were selected. A similar technique was used for calculating cut-off value of the correlation coefficient: (a) the frequency distribution of the correlation coefficient was computed across profiles of all 14 variables analyzed; and (b) the value of the 75^{th} percentile (0.91) was accepted as a cut-off to eliminate profiles which did not fit a model well. As a result, seven profiles were eliminated where the cosine function period was longer than the observation time (14 days). Distinct rhythms were evident for the time-dependent fluctuation (days) of the corresponding plasma cytokine concentrations/cell counts. Table 4 shows the periods in days of the eight cosine curves which satisfied the selection criteria in this example. The shortest period is 3 days and all other periods except one are multiples of 3: 6, 9 and 12. One exception in this example is a 4 day period of IL12p70 in patient 1.

TABLE 4

| Patient | | CD197/ | IL- | | CRP | CD11c/ | | CD4/ |
|---|---|---|---|---|---|---|---|---|
| number | PFS | CD206 | 12p(70) | IL-17 | ng/mL | 14 | IL-1ra | 294 |
| 1 | 916 | 6 | 4 | | | | | |
| 4 | 748 | | | | | | | |
| 6 | 132 | | | | | 3 | | |
| 5 | 91 | | | | | | | 4 |
| 12 | 77 | | | 12 | | | 4 | |
| 10 | 70 | 12 | | | | | | |
| 7 | 68 | | | | | | | 3 |
| 2 | 37 | 3 | | 6 | 9 | | | |

The data in Table 4 show that distinct rhythms were evident for the time-dependent fluctuation (days) of the corresponding plasma cytokine concentrations/cell counts, specifically the ratio of polarized M1/M2 macrophages (CD197/CD206) (30), Interleukin-12 (IL-12p70), Interleukin-17 (IL-17), C-reactive protein CRP), CD11c-positive monocytes (CD11c/14) and Th2 helper T lymphocyte cell subset (CD4/294). For the majority of patients/variables, these rhythms followed a predictable pattern which was a multiple of 3 days (3, 6, 9 and 12 days, respectively) for most of plasma cytokines and cell counts. A few patients demonstrated a 4 day periodicity for IL-12p70, IL-1ra and CD4/294.

Determining the number and frequency of blood draws needed to accurately detect sinusoidal fluctuations in immune variables. The extrapolation of the obtained curves (FIG. 1C) for the time length of two periods (6, 12, 18 and 24 days correspondingly) demonstrated that every day sampling for at least 24 days would achieve an R square of 0.9 for cosine curve fitting. A data series collected with this frequency and for this period of time may allow more reliable analysis of the dynamics of those variables which fluctuate with amplitude not less then 45% of the mean value of the variable during the whole time of the observation (24 days). Only time-dependent concentration profiles with periods 12 days or shorter may be reliably analyzed under the described conditions. This analysis outlines the parameters of study design (frequency and duration of sample collection) necessary to directly test the hypothesis of the impact of timed chemotherapy delivery based on fluctuating immune variables (ongoing validation study).

Referring again to FIG. 1C, once the process extrapolates values for each of the selected immune variables, the process may compute the date(s) when the product Π achieves it's maximum values for each of the selected immune variables within the extrapolated time period (142). The process next computes the dates when the maximum number of immune variables will have maximum values of the product Π (144). The process may report dates when the maximum number of immune variables will have maximum values of the product Π (146). These dates may correspond to a proposed day of treatment that has the best correlation with the patient's PFS.

The process may also output a report/table/plot of extrapolated and/or maximum values of Π products per variable for a period of 24 days after the last measurement, output a table of ranks or products per immune variable and output a plot of maximum values of product Π per variable for a period of 24 days after the last measurement (148).

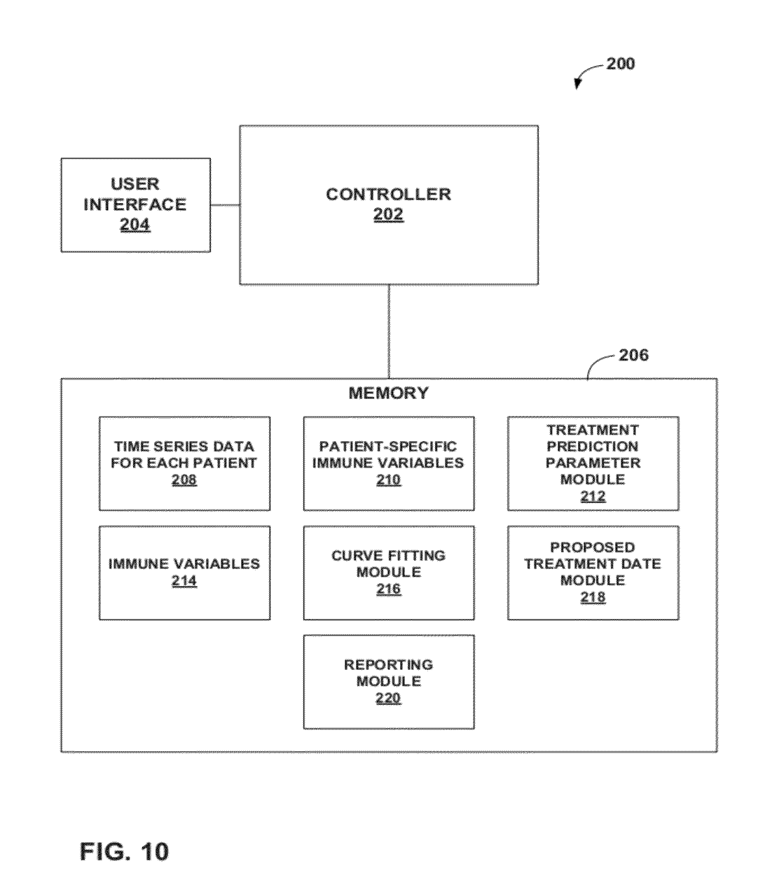

FIG. 10 is a block diagram illustrating an example system 200 for determination of favorable times for delivery of chemotherapy treatment. The system includes a controller 202 which processes the data and determines predicated favorable treatment times based on the biological parameter data for one or more patients. The system also includes a user interface 204 through which a user may input various process parameters and/or may view reports of the results of the analysis of time series data for one or more patients. The results may be presented in report format, and may include text, plots, graphs, charts, or other meaningful way of presenting the results. The user interface may also permit a user to input process parameters and/or data to be used by the system. A memory 206 stores the data and programming modules needed to analyze the time series data for one or more patients. For example, the memory may store the time series data for one or more patients 208, a list of the potential immune variables 214, and the patient-specific immune variables that fit a periodic function for that patient 210. The memory may also include a treatment prediction parameter module 212, a curve fitting module 216, a proposed treatment date module 218, and a reporting module 220 may generate reports regarding each patient's predicted favorable treatment times. These reports may be printed, transmitted to a local or remote computer and/or displayed on a local or remote computer.

The memory may also include programming modules such as a curve fitting module, a reporting module, a treatment prediction parameter (Π) module and a proposed treatment date module. Curve fitting module receives time series data of immune variable concentration for an observed time period for each of a plurality of identified immune variables and fits a periodic function to the time series data corresponding to each of the plurality of identified immune variables. Treatment prediction parameter module performs all of the calculations necessary to determine the treatment prediction parameter (Π), such as defining a relative concentration of the fitted periodic function, defining a relative derivative of the fitted periodic function and calculating the treatment prediction parameter based on the relative concentration and the relative differential.

Proposed treatment date module may choose the proposed treatment date such that the treatment prediction parameter (Π) is maximized. Reporting module may generate screen displays or printable reports including the proposed date of treatment that maximizes the treatment prediction parameter and/or other presentations of the raw data, intermediate data, or final results. The reporting module may allow the user to create customized reports depending upon the format and/or data the user wishes to view.

The system shown in FIG. 10 also includes a controller that, by following the programming modules stored in the memory, analyzes the time series data and determines proposed dates for timed delivery of chemotherapy as described herein.

The example study discussed herein describes the time-dependent (kinetic) relationship between the tumor and host immune response in 10 patients with metastatic malignant melanoma. The data analysis suggested that most biomarkers show a temporal variation, implying that these immune variables oscillate repeatedly, in an apparent predictable fashion. This is consistent with previously published reports of episodic “rhythmic” changes in hematology and immunobiology which follow a circadian (24 hour), infradian (greater than 24 hours—for example seven days or circaseptan), seasonal, or circannual (yearly) pattern. The use of single time point studies to describe the state of immune homeostasis in patients with cancer may be overly simplistic and potentially misleading. Therefore, the temporal variation of measured biomarkers and the pattern of change (and not only the degree of change itself) may better define an individual's response to illness.

The techniques described herein may provide evidence that rhythms exist in immune responses to malignant disease and suggest the possibility that such rhythms may be relevant to therapeutic success. Disruption of such biorhythms may have clinical consequences. These observations are consistent with the findings that patients with disorganized (non-curve-fitting) anti-tumor immune responses (see, e.g., FIG. 3) experienced a significantly decreased survival (PFS of 71 and 74 days, respectively), relative to those in whom the measured immune variables followed a predictable biorhythm (coefficient of correlation 0.72). In this example, it appeared that best clinical outcomes were observed in the two patients who best maintained a well synchronized anti-tumor immune response possibly overcoming global immune dysfunction of malignancy. Timed delivery of chemotherapy in that context may have allowed for a more precise therapeutic intervention leading to putative depletion of immune down-regulatory signals in favor of effective anti-tumor immunity.

In this example, distinct infradian rhythms were found in the fluctuations of most variables fitted to cosine functions which were in fact multiples of 3-4 days. The contribution of circadian variation to the fluctuation of immune variables was minimized in the example study by collection of blood samples at approximately the same time of day (between 8 and 10 AM); therefore the rhythms observed in the example study are unlikely to be influenced by daytime/nighttime schedule.

By extrapolating the principle of chronotherapy to the anti-tumor immune response, it is possible that coupling treatment with these rhythms will improve the therapeutic index of cancer chemotherapy. It was originally posited that timed application of chemotherapy at a certain point in the immune cycle, based on the fluctuation of the CRP concentration, could selectively ablate the cycling suppressive elements of immunity, thus releasing the patient's immune system from down-regulation. However, the data (such as that presented herein) demonstrated no significant correlation between PFS and CRP concentration on the day of treatment. In this example, in order to accurately predict the fluctuation of the immune response and successfully time chemotherapy administration, one needs to consider not only the magnitude of change in concentration or immune cell frequency but also the dynamic change of a particular immune variable. In order to better characterize this time-dependent change, the analysis was extended to 29 other cytokines/chemokines/growth factors and 22 immune cell subsets and studied 1593 additional data points measured over 15 days in 10 patients with metastatic melanoma. By using mathematical modeling and curve fitting analysis a single parameter (Π) was defined that describes both the magnitude of change in concentration and the trend for increase or decrease of a given immune biomarker. This parameter may then be used to identify the variables for which application of chemotherapy at a distinct time-point in the immune cycle correlated with improved PFS.

CRP was initially an attractive candidate given its well established quantification methodology, ease of measurement, as well as previously described periodic fluctuations in healthy individuals as well as patients with chronic viral infections or cancer. The example data analysis, however, showed that there may be no correlation between CRP changes and clinical outcome (PFS) (correlation coefficient −0.60). Unexpectedly, two other variables, concentration of IL12p70 and the ratio of CD197/CD206 positive cells (ratio of polarized M1/M2 macrophages) exhibited satisfactory correlation with PFS in these examples, emerging as potential candidate biomarkers for timed administration of chemotherapy. Other biological variables, including some of those described herein, may also be appropriate biomarkers, depending at least in part upon the patient.

It shall therefore be understood that other immune variables not described herein may also, upon further study, exhibit satisfactory correlation with PFS, and that the disclosure is not limited in this respect.

The example study described herein shows that IL-12 fluctuates in a predictable pattern in patients with cancer (4 day period) and that application of TMZ therapy at a particular time-point when IL-12 is at a concentration peak or shows a strong positive trend (positive first derivative of the fitted function) may result in enhanced treatment effect and improved clinical outcome. The additional immunomodulatory properties of TMZ (in addition to its anti-tumor activity) may augment immunological responsiveness through destruction of regulatory T cells, disruption of homeostatic T cell regulation, or abrogation of other inhibitory mechanisms. Timed administration of this agent at a particular time-point in the immune response cycle when IL-12 shows a positive trend (2 out of the 4 day period), may selectively suppress Treg who lag behind T effectors in their clonotypic expansion. By that time, effector T cells may have proliferated and become activated and may be therefore less susceptible to the effects of TMZ chemotherapy.

In the example described herein, curve simulations using function parameters obtained in nonlinear regression fitting of cosine curves to the sample data with periods of 3 to 4 days. This simulation sought to (a) further assess the significance of curve fitting to experimental data; and (b) get a more accurate estimate of the minimum number of data points sufficient for reliable curve fitting, which may allow better planning for a future clinical trial.

Based on the extended example simulation data, an example list of candidate biomarkers, may include, for example, CRP, IL-10, IL-12p70, G-CSF, IL-9, VEGF, IL-1ra, IL-13, IL-15, IL-17, and immune cell subsets such as CD4/294, CD11c/14, CD197/CD206, CD206 and DR(hi).

In summary the data suggests that: (a) patients with stage IV melanoma exhibit a dynamic, not static, anti-tumor immune response; (b) an ordered pattern of change in plasma concentration of various cytokines/chemokines/growth factors and immune cell subsets was observed in patients with the longest PFS; (c) the fluctuations of most variables fit cosine functions with periods which are multiples of 3-4 days; and (d) delivery of cytotoxic therapy (TMZ) at a defined time in the biorhythmic immune oscillation appears to correlate with improved clinical outcome. The product between the relative concentration of an immune variable and the first derivative takes into consideration both the magnitude of the concentration and the dynamic trend of a given variable and could be used to guide personalized “timed” drug delivery. The data presented herein provide the basis for the design of experimental conditions for testing the hypothesis of timed chemotherapy delivery at a specific phase of the immune cycle.

In a more specific example, a cosine curve simulator (CCS) software module generates simulated cosine/sine curves using function parameters obtained in experiments measuring time-dependent concentration of a selected group of proteins in human blood samples. As discussed above, the simulator takes as an input time series measurements of concentrations of biological variables samples drawn from a number of patients. The other input is distribution of frequencies of technical errors of various magnitudes which was also measured in the experiment. The software outputs curves corresponding to 9 mathematical functions fitted to the input data series. Each fitted curve is supplemented with goodness of fit parameters. The software also outputs a table and a plot of probabilities of cosine curve detection as related to the amplitude, function period, frequency of sampling and length of the observation period.

One purpose of the CCS is to assess confidence bounds of the parameters of the data sets (period of observation, frequency of blood sampling, range of detectable periods of concentration fluctuation, range of detectable amplitudes of concentration fluctuation) for detection of data fitting to 9 mathematical functions.

The CCS algorithm may receive input as described above. The average value and standard deviation is calculated for each biological variable (concentration of a cytokine, chemokine, growth factor or a cell count of a specific cell type) across samples. A range of average +/−2 standard deviations is calculated for each parameter in the cosine function. There are 4 parameters in the cosine function f(x)=A+B*cos(C*x+D): parameter A determines the vertical shift of the curve, parameter B determines the amplitude, parameter C determines the period and D defines phase shift.

In one example, the range for parameter B is divided into 100 increments, and range for parameter C is divided into 20 increments to produce periods in the range from 1 to 20 days with 1 day increment. The CCS simulates a set of data points (which correspond to concentration of a protein or cell count) for all possible combinations of period and amplitude for each variable. Further, data may be simulated for three periods of observation: 10 day, 15 days and 20 days and for three frequencies of blood sampling: every day, every other day and with 1 to 2 day interval. Such a simulation will generate 936,000 data sets in total (52 variables*100 amplitudes*20 periods*3 observation periods*3 sampling frequencies). Collectively these data sets may be referred to as “Series A”. A signed experimental error is be added to the ideal value of the function. The error value and frequency follows the distribution of error values obtained in the experiment and the sign is random.

R squared (R²) and standard error may be calculated for each simulated data set. The CCS generates a table and a histogram of distribution of frequencies of R². Further, CSS may generate another series of data sets—“Series B”. Each set of data points in this series may have the same combination of parameters (52 combinations of amplitude, period, observation period, sampling frequency. One combination per biological variable). However, in this example, the value of the function is not calculated by the cosine formula, but rather is a random number. This random number satisfies all above named parameters.

The curve-fitting as described above may then be applied to the simulated data. For example, curve-fitting may be applied to each data set to 9 mathematical functions (linear function, exponential function, exponential association, logistic model, Morgan-Mercer-Flodin (MMF) model, quadratic function, cosine function, rational function, Gaussian model) and reports which data sets fit any of the functions with R squared above 75^{th} percentile cut-off. The list of these data sets (IDs) may then uploaded into the CCS. Using “Series B” the CCS computes p-value for each simulated data set from the uploaded list. CSS outputs a table of simulated datasets with their parameters and associated p-values. These p-values represent the probability that a data set with a given combination of parameters is fitted uniquely to a cosine curve by chance alone.

A common problem for mathematical modeling of clinical data is the limited number of data points. Developing a model of a dynamic process requires a time series of measurements. Translated into the terms of a clinical setting this means blood or tissue samples collected with certain frequency over some period of time. It is common that the frequency and observation period allowed by the clinical standards are not sufficient to develop a mathematically sound model. For example, fitting protein concentration in blood measured six times during a period of two weeks to a cosine curve produces ambiguous results. Simulation and modeling study allows one to define experimental parameters to more reliably determine the function of a dynamic trend.

Fitting of 6 or 7 data points to a function with four parameters (sinusoidal and rational functions) is ambiguous even if the goodness-of-fit metrics are satisfactory (R² and coefficient of variation are close to 1.0, confidence interval is narrow, etc.). A straightforward way to resolve this ambiguity is to increase the number of data points. However, in a clinical setting this solution has strict limitations. In many situations human samples (blood or tissue) cannot be collected for long enough periods of time and frequently enough to obtain a time series of data points which would unambiguously satisfy stringent curve fitting criteria.

The techniques described herein may also determine sampling frequency, observation period, curve amplitude and period for one or more biological parameters that fit a function to within a desired goodness of fit. These sampling parameters may then be used to determine a schedule for the real-world collection of blood or tissue samples from patients that will be sufficient to adequately determine desired treatment times. Such a sample collection schedule results in a sufficient number of time points to arrive at a sufficiently accurate determination of desired treatment times while keeping the burden for patients as low as possible. In other words, given the maximum possible number of data points, determine sampling frequency, observation period, curve amplitude and period (for periodical function) which fit a function with high probability not by chance alone.

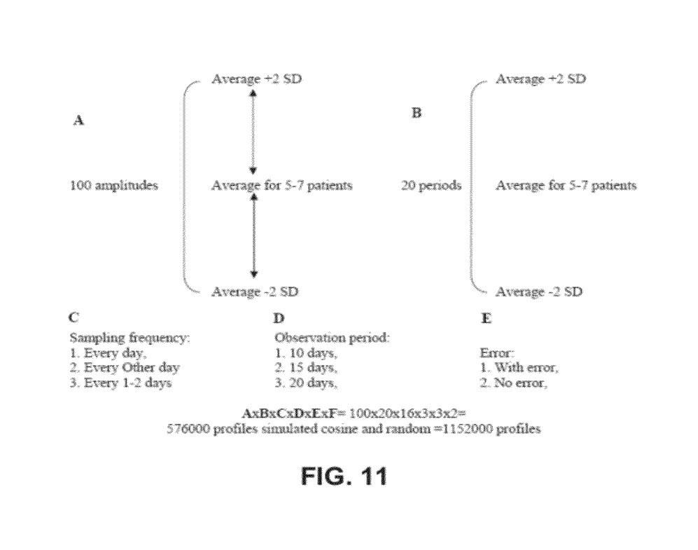

Time series of data points were simulated with input parameters derived from the example clinical data. FIG. 11 illustrates an example simulation which considered three different observation periods (10, 15 and 20 days), three various sampling frequency (every day, every other day and 1-2 days), one hundred amplitudes and twenty periods. In the example study, the following variables fitted cosine curves by defined selection criteria and had periods equal or shorter than 12 days: CD197/CD206 and IL12p70 (5 patients); CD4/294 and IL-15 (4 patients); CRP, IL-10, CD11 c/14, CD206, IL-17, IL-13 (3 patients); IL-1ra, 11-9, G-CSF and VEGF (2 patients) and DR(hi) (one patient). Taking this into account, the amplitudes for a given variable were simulated as follows. The average of the parameter B, which defines the amplitude of the cosine function, was calculated across all patients in whom the time series for the variable fitted cosine curve. The interval B_{arg}+/−two standard deviations was calculated and divided into 100 fragments (see, e.g., FIG. 11.). Each of the 100 values of parameter B was used in the cosine equation to produce a profile with specific amplitude. Twenty different periods were simulated by the same technique. Each data series was simulated with or without experimental error. The error was calculated from the values of coefficient of variation maintaining the same distribution of error values as was obtained in the experiment. The error was added to or subtracted from the simulated value in random order. Time series for 16 variables which fitted cosine curve with R² above the 80 percentile cut-off in at least 7 out of 8 patients were simulated. Two sets of time series were simulated according to the described design. In the first set (Cosine profiles) concentration/cell count values were calculated by the cosine formula. In the second set (Random profiles) values were produced by the generator of random numbers within the set amplitude range. As result, 576000 data series of cosine profiles and 576000 data series of random profiles were obtained. All these profiles were fitted to the following five functions: logistic function, quadratic function, cosine function, rational function, Gaussian function, and MMF function (Morgan-Mercer-Flodin) and R² was recorded for each fitting.

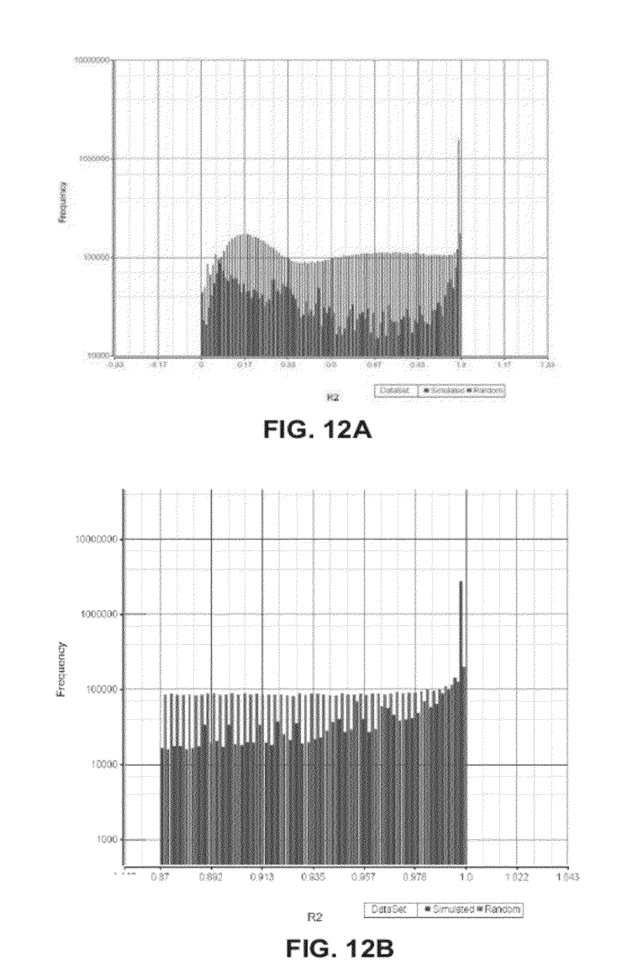

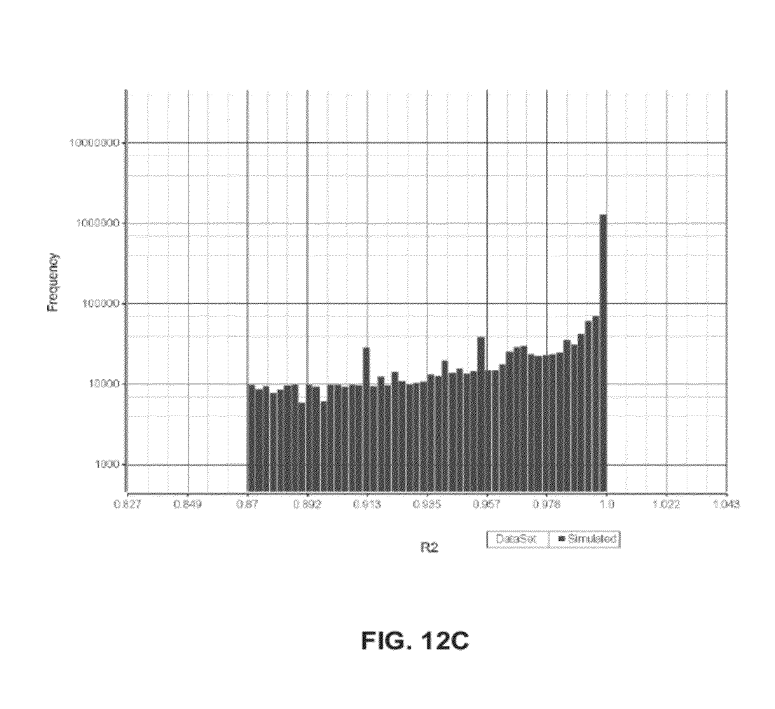

FIGS. 12A-12C are graphs illustrating the frequency distribution of R2 for various ranges and datasets. To determine potential clinical schedules for collection of data that would result in sufficiently accurate determination of desired treatment times, the proposed clinical schedules with multiple combinations of parameters were analyzed. The distribution of R² of the curve fitting in random and cosine data sets (see FIG. 12A) was computed and analyzed. Since the most of time series of measurements in original experiment fitted cosine curve, the properties of R² distribution for cosine function will now be described. The analysis of the R² distribution may permit identification of conditions (period, amplitude, sampling frequency, observation period, etc.) which predominantly produce true positive and true negative solutions as well as those which produce false positive and false negative solutions. A solution is the conclusion whether or not a time series of data points fits a cosine curve based on the value of R². Simulated profiles computed by the cosine formula produced true positive and false negative solutions when R² was high or low correspondingly. Likewise, random profiles produced false positive and true negative solutions. As a result, ranges of R² values corresponding to high sensitivity and specificity of the solutions can be determined One of the goals of the simulation study was to determine the cutoff values of R² which allow one to achieve best combination of specificity and sensitivity.

A small number of time series (10185 profiles=0.0088% of the total number of profiles) formed straight lines and were excluded from further analysis. For the cosine profiles, about 81.7% (461998 out of 565821) of R² values lie in the range 0.980-1.0 (FIG. 12B). Of those, values obtained from fitting data series without introducing an error comprised 50%. The 90^{th} percentile of the R² values for the cosine profiles was 1.0 and 0.905 for the random profiles. The overall 90^{th} percentile of the R² values in the range from 0 to 0.98 was 0.87. R² values in the range from 0.87 to 1.0 were then considered. In one example, it may be reasonable to use the 90^{th} percentile of R2 subset as cut-off criteria for discriminating between random set of data points and those calculated by the cosine formula. This cutoff (rather than a more stringent 0.98) prevents having a larger number of false negative results. In other examples, other appropriate R2 cutoff could be used. The resulting subset of R² values contains ambiguous solutions (false positives and false negatives), the majority of which are introduced by profiles generated with observation period of 10 days and every other day blood sampling frequency. When all profiles generated with both of these conditions are removed, then only simulated cosine profiles fit cosine function with R² in the interval 0.8995 to 0.995 (FIG. 12C). No other tested observation period or sampling frequency produces significant number of R2 in this interval from random profiles.

As expected, the proportion of R² above the 90^{th} percentile cut-off obtained from fitting cosine profiles is higher for profiles with greater number of time points, that is, longer observation period or frequent blood sampling. This is a limiting factor in a clinical trial because blood samples cannot be taken during a long period of time with high frequency. This calls for an experimental design which would be a compromise between clinical requirements and demands of the curve fitting methods. Such a design is a sample collection schedule which allows a sufficient number of time points but keep the burden for patients as low as possible. A schedule satisfying these conditions is 5 sequential days when blood samples are collected, then 2 days of rest followed by another 5 days of sample collection. Such a collection schedule will be referred to herein as the “5-2-5 schedule.”

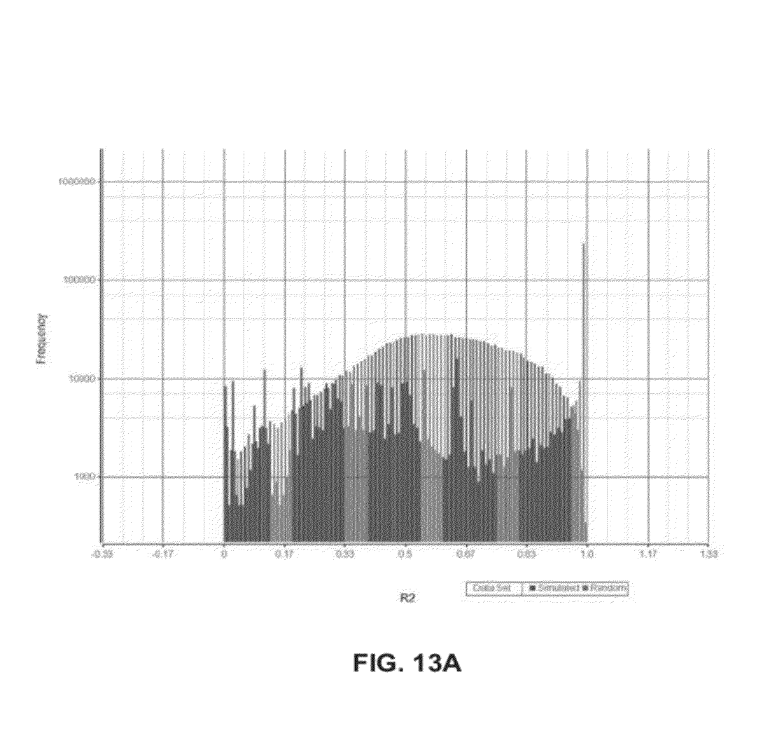

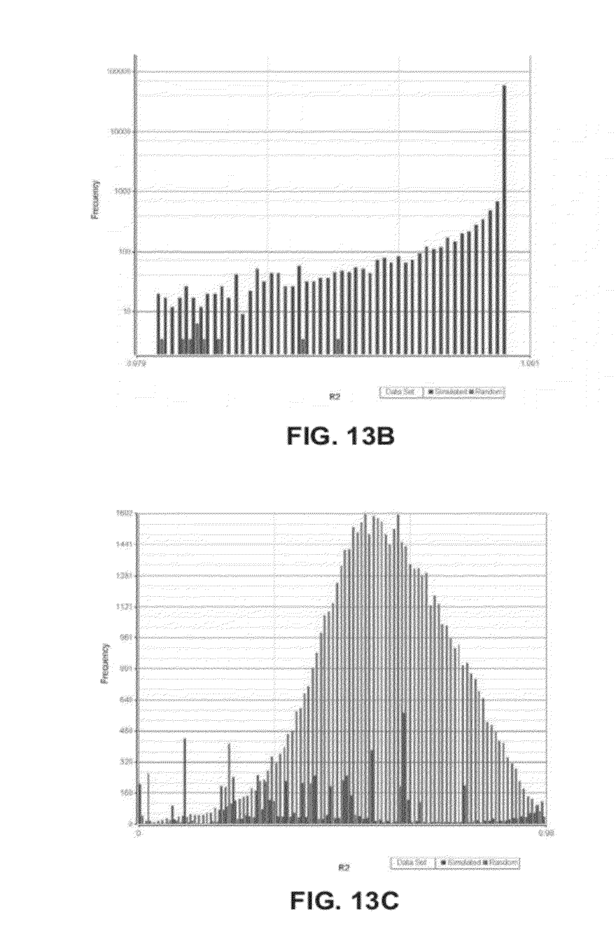

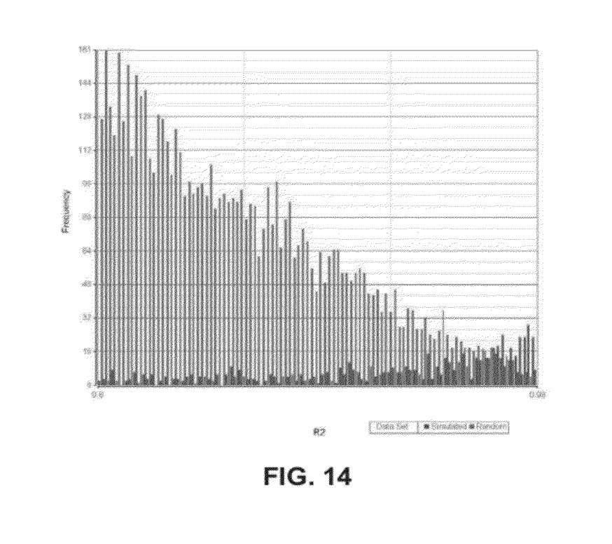

The 5-2-5 schedule gives 6 degrees of freedom for data fitting to a cosine function. Time series were simulated for this schedule. FIGS. 13A-13C are graphs illustrating the frequency distribution of R² for an example simulated 5-2-5 sample collection schedule. All R² values (56119 out of 56128) above 0.980 were generated by fitting simulated cosine profiles (FIG. 13A). The R² obtained from fitting the random profiles to the cosine function were largely prevalent in the range 0.000-0.980. The distribution of R² in this range is quasi-normal (FIG. 13C). The 90^{th} percentile of the subset of R² values in the range from 0 to 0.980 is 0.8055 (FIG. 13B). It follows, that if 90^{th} percentile is selected as a cut-off criteria for discriminating between random set of data points and those calculated by the cosine formula, then ambiguous solutions will lie in the R² value range from 0.8055 to 0.980 (FIG. 14). The receiver operating characteristic (ROC) analysis of 16 variables for this interval of R² values was determined The best performing variable was IL-1ra (area under the curve (AUC)=0.955) and the worst performing variable was CRP (AUC=0.734) as shown in Table 5.

TABLE 5

| Variable | AUC |
|---|---|
| IL-1ra | 0.955 |
| IL-17 | 0.91 |
| CD197/CD206 | 0.886 |
| IL-9 | 0.884 |
| VEGF | 0.875 |
| CD11c/14 | 0.856 |
| IL-12p70 | 0.854 |
| CD206 | 0.844 |
| IL-10 | 0.844 |
| IL-13 | 0.837 |
| G-CSF | 0.824 |
| CD11c/CD123 | 0.806 |
| CD4/294 | 0.795 |
| IL-15 | 0.785 |
| DR(hi) | 0.778 |
| CRP | 0.734 |

Since the hypothesis in this example was that maximums of index Π indicate active state of the immune response to tumorigenesis which is favorable for therapeutic treatment, the process may identify time periods when maximum number of variables have maximum cumulative value of index Π. The process may account for variability of periods, increase and decrease rate of the change of immune variables such as concentration and cell counts as well as variability of the amplitude. Considering the intrinsic flexibility of a biological system in general, time periods corresponding to the set properties of immune parameters may be determined as intervals of time when the probability that immune parameters satisfy the set properties is elevated. Time intervals may be determined within the observation period as well as predicted in the future. The probability may gradually diminish in the vicinity of its peak value following normal or non-normal distribution.

Various methods can be used to identify the time periods of increased probability. In one example, a clustering algorithm, such as modified K-means clustering or other clustering algorithm, may be applied to find these time intervals for the time series generated in the 5-2-5 simulation. In this example, this method identified two days within a 12 day observation period when the cumulative index had maximum value. The same analysis may then be performed on the data obtained from patients with long PFS (916 days; Patient #1 and 841 days; patient #4) and short PFS (68 days; Patient #7 and 70 days Patient #10). Time series of three variables were clustered: concentration profiles of IL-1ra, IL-12p70 and counts of CD206⁺ cells for these four patients. Since time series obtained from the clinical trial had only 7 or 6 data points, 3 or 4 additional data points were extrapolated to match the same number of points (10) as were analyzed in the simulated 5+2+5 data set. The extrapolated values were computed using Fourier analysis. Clustering produced 1-3 days with maximum cumulative value of index Π for each patient, as shown in Table 6. In another example, Markov Chain Monte Carlo (MCMC) method can be applied to identify time intervals when the probability that immune parameters satisfy the set properties is elevated. In this case, the random walk step of the MCMC is used to find the sought time intervals at a future time. In yet other examples, Bayesian methods or Multiobjective optimization can be applied to find these time intervals. It shall be understood, therefore, that the disclosure is not limited in this respect.

TABLE 6

| | | | Days | Minimum difference |
|---|---|---|---|---|
| Patient | | Treatment | predicted by | between treatment and |
| number | PFS | day | clustering | clustering days |
| 1 | 916 | 18 | 6, 21 | −3 |
| 4 | 841 | 11 | 13.5; 8.5 | −2.5 |
| 7 | 68 | 14 | 3.2; 9.8; 20.5 | 4.2 |
| 10 | 70 | 15 | 6.14 | 8.9 |
| 2 | 37 | 12 | 14, 6, 0.5 | −2 |
| 5 | 91 | 14 | 8.6 | 5.4 |
| 6 | 32 | 17 | 8.1 | 9 |
| 12 | 77 | 20 | 5.1, 24.2 | −4 |

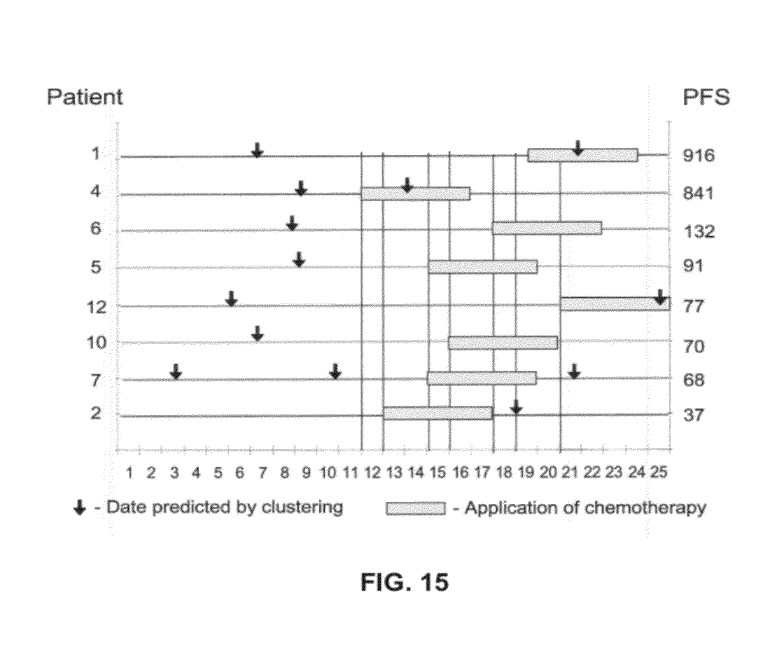

FIG. 15 is a chart illustrating the association between the 5-day period of actual chemotherapy application, time predicted by the example clustering algorithm and PFS in 8 melanoma patients. An example clustering method was applied to preliminary data obtained in a pre-clinical trial on 8 stage IV melanoma patients. Progression-free survival (PFS) time varied from 37 days to 916 days in these patients. Favorable time for chemotherapy application predicted with by the clustering algorithm fell within the 5-day period of chemotherapy application in two patients with the longest PFS (Patients #1 and #4). In all other patients except one, chemotherapy was applied several days before or after the days predicted by the clustering. In one patient, the day predicted by the algorithm fell on the last day of chemotherapy application (Patient #12).

It is noteworthy that treatment days were very close to the days identified by clustering in patients who had long PFS (Patients #1 and #4 in FIG. 15). In patients with relatively shorter PFS the treatment was delivered 6.6 (Patient #7) and 8.5 (Patient #10) days earlier than predicted by clustering (FIG. 15). Only profiles which fit cosine function with correlation coefficient greater than 0.86 were used. Based on this criterion IL-1ra was eliminated from clustering in Patients #1, 4 and 7 and the IL-12p70 profile was eliminated in Patient #10.

The techniques described herein for selecting one or more immune variables which may be as predictors of patient's response to pharmaceutical treatment, such as chemotherapy. The basic principle of the method is to accumulate and analyze the knowledge on performance of each of the measured variables in each patient in whom the measurements and the treatment were performed. This accumulation is achieved through creation of a database in which time series of measurements and progression-free survival (RFS) time are recorded. In some examples, the algorithm computes and enters into the database the R² value of the fitting of each time series to the cosine function. Next, frequency distribution of R² values is computed and the R² value of the 75^{th} percentile may be defined. This value serves as a cut-off for selecting variables in the next steps of the algorithm. Depending on required stringency of variable selection, a higher (or lower) R² cut-off level can be selected, for example, 80^{th} or 90^{th} percentile (or lower than 75^{th} percentile).

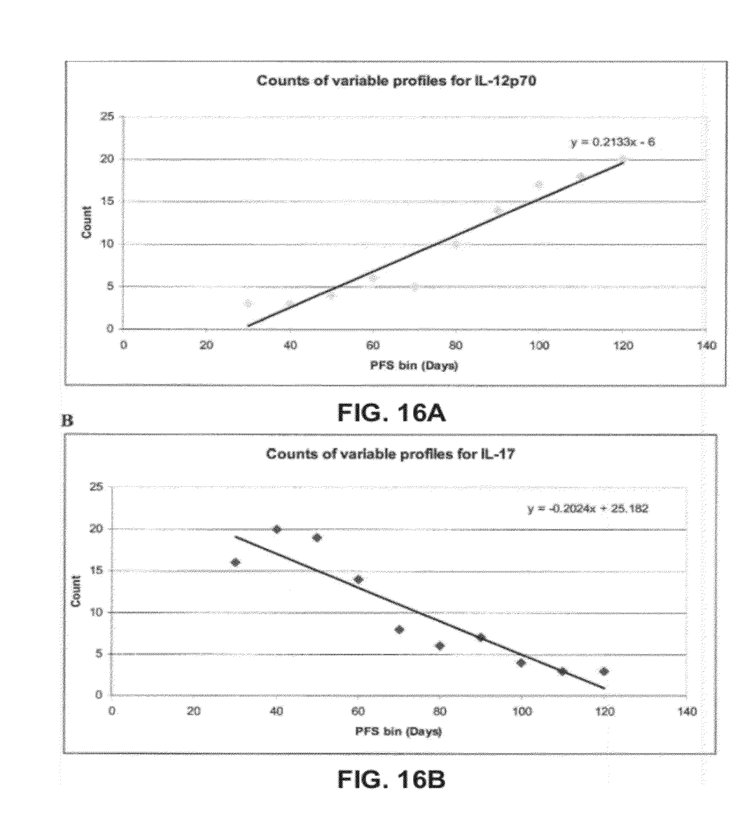

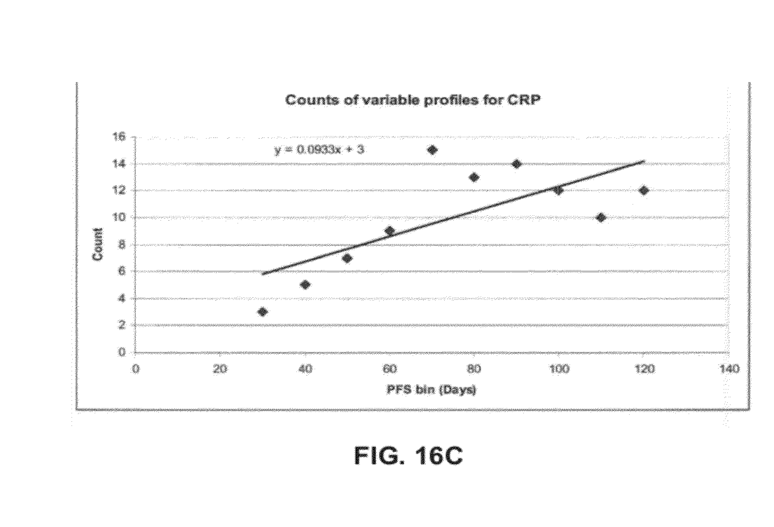

In another example, in order to select immune variables to be used as discriminators in the clustering algorithm, the algorithm may divide the whole range of PFS longevities into the number of bins ten times less than the number of patients. For each bin the algorithm counts profiles of each variable with R² above the cut-off value and the sum of Π indices on the treatment start date for these variables (see, e.g., Table 7 and Table 8). Next, the linear regression analysis is performed both on the counts of each variable with R² above the cut-off value and on the sums of Π indices and the slope of the regression line is computed. Variables with high positive value of the sum of the slopes (for example, IL-12, IL-1ra and CD206 in Table 7) have positive correlation (PC) with PFS (see, e.g., the graph for IL-12p70 in FIG. 16A), variables with high negative sum of the slopes (for example, IL-17 and IL-10 in Table 7) have negative correlation (NEC) (see, e.g., the graph for IL-17 in FIG. 16B), and variables with sum of the slopes close to zero (for example, IL-13, IL-15 and CRP in Table 7) have no correlation (NOC) with PFS (see, e.g., the graph for CRP in FIG. 16C). In this example, the cut-off for PC variables is the 75^{th} percentile (mean+0.67×Standard Deviation) of all sum values and for the NEC the cut-off is the 25^{th} percentile (mean−0.67×Standard Deviation). Alternatively, to decrease the stringency of the variable selection either cut-off of the slopes for only regression line of the counts, or only slopes for sums of Π indices can be considered.

TABLE 7

| | | | | | | | | | | | Num of | | | |
|---|---|---|---|---|---|---|---|---|---|---|---|---|---|---|
| Variable ↓ | Counts of variable profiles | | | | | | | | | | patients | Slope | Mean | SD |
| IL-12 | 3 | 3 | 4 | 6 | 5 | 10 | 14 | 17 | 18 | 20 | 100 | 2.13 | 0.51 | 1.80 |
| IL-13 | 8 | 9 | 10 | 6 | 8 | 15 | 10 | 12 | 9 | 13 | 100 | 0.45 | 75^{th} percentile | |
| IL-15 | 8 | 13 | 10 | 9 | 10 | 12 | 9 | 12 | 8 | 9 | 100 | −0.09 | 1.72 | |
| IL-17 | 16 | 20 | 19 | 14 | 8 | 6 | 7 | 4 | 3 | 3 | 100 | −2.02 | | |
| IL-10 | 18 | 20 | 16 | 12 | 10 | 8 | 6 | 3 | 3 | 4 | 100 | −2.00 | | |
| IL-1ra | 2 | 3 | 4 | 3 | 8 | 9 | 10 | 19 | 22 | 20 | 100 | 2.37 | | |
| CD206 | 2 | 2 | 4 | 5 | 7 | 10 | 12 | 17 | 20 | 21 | 100 | 2.34 | 25^{th} percentile | |
| CRP | 3 | 5 | 7 | 9 | 15 | 13 | 14 | 12 | 10 | 12 | 100 | 0.93 | −0.69 | |
| PFS bin→ | 30 | 40 | 50 | 60 | 70 | 80 | 90 | 100 | 110 | 120 | | | | |

TABLE 8

| Variable ↓ | Sum of π indices | | | | | | | | | | Total | Slope | Mean | SD |
|---|---|---|---|---|---|---|---|---|---|---|---|---|---|---|
| IL-12 | 10 | 11 | 17 | 19 | 33 | 62 | 74 | 93 | 138 | 157 | 614 | 16.90 | .63 | 2.60 |
| IL-13 | 12 | 17 | 22 | 34 | 27 | 42 | 67 | 87 | 112 | 142 | 562 | 13.78 | 75^{th} percentile | |
| IL-15 | 15 | 14 | 23 | 17 | 19 | 21 | 16 | 20 | 18 | 19 | 182 | 0.29 | 13.07 | |
| IL-17 | 196 | 173 | 152 | 163 | 110 | 83 | 63 | 54 | 37 | 22 | 1053 | −20.20 | | |
| IL-10 | 63 | 67 | 54 | 57 | 62 | 68 | 59 | 57 | 61 | 64 | 612 | −0.04 | | |
| IL-1ra | 34 | 47 | 59 | 72 | 84 | 98 | 124 | 157 | 178 | 205 | 1058 | 18.90 | | |
| CD206 | 13 | 12 | 17 | 22 | 26 | 32 | 43 | 52 | 57 | 68 | 342 | 6.40 | 25^{th} percentile | |
| CRP | 23 | 34 | 42 | 54 | 52 | 48 | 53 | 47 | 41 | 34 | 428 | 1.00 | −3.81 | |
| PFS bin→ | 30 | 40 | 50 | 60 | 70 | 80 | 90 | 100 | 110 | 120 | | | | |

TABLE 9

| | Slope for the | Slope for | | | |
|---|---|---|---|---|---|
| | number of | the sum of | | | |
| Variable | counts | PI | Sum | Mean | SD |
| IL-12 | 2.13 | 16.90 | 19.03 | 5.14 | 14.0 |

| | | | | |
|---|---|---|---|---|
| IL-13 | 0.45 | 13.78 | 14.23 | 75^{th} percentile |
| IL-15 | −0.09 | 0.29 | 0.21 | 14.56 |
| IL-17 | −2.02 | −20.20 | −22.22 | |
| IL-10 | −2.00 | −0.04 | −2.04 | |
| IL-1ra | 2.37 | 18.90 | 21.27 | |
| CD206 | 2.34 | 6.40 | 8.74 | 25^{th} percentile |
| CRP | 0.93 | 1.00 | 1.93 | −4.28 |

Tables 7-9 illustrate data corresponding to example procedures that may be used to select immune variables that will may used as discriminators in the clustering algorithm. The range of PFS time is divided into a number of bins (clusters) 10 times less than the number of patients. In this example there were 100 patients and so the PFS times were divided into 10 PFS bins (see, e.g., the last row of Table 7).

Temporal profiles which fit the cosine function with R² greater than selected cut-off are counted for each RFS bin and the slope of the regression curve of the counts is computed. Table 7 shows the mean and standard deviation (SD) of the slope values for all variables. These are used to calculate the 75^{th} percentile (mean+0.67×Standard Deviation) and the 25^{th} percentile (mean−0.67×Standard Deviation) of the slope values. In this example, variables for which the slope values were above the 75^{th} percentile include IL-12, IL-1ra, and CD206. Variables for which the slope values were below the 25^{th} percentile include IL-17 and IL-10.

Table 8 shows the sums of Π indices on the first treatment day for temporal profiles which fit the cosine function with R² greater than selected cut-off are computed for each RFS bin and the slope of the regression curve of the sums is computed. The mean and standard deviation (SD) of the slope values for all variables are computed and are used to calculate the 75^{th} percentile (mean+0.67×Standard Deviation) and the 25^{th} percentile (mean−0.67×Standard Deviation) of the slope values. In this example, variables for which the slope values were above the 75^{th} percentile include IL-12, IL-13 and IL-1ra. Variables for which the slope was below the 25^{th} percentile include IL-17.

Table 9 shows the sum of the slope values computed in Table 7 and Table 8 for each variable. The mean and standard deviation (SD) of the sums for all variables are computed and are used to calculate the 75^{th} percentile (pink) and the 25^{th} percentile (blue) of the slope values. In this example, variables for which the sum of the two slope values were above the 75^{th} percentile include IL-12 and IL-1ra. Variables for which the sum of the two slopes that were below the 25^{th} percentile include IL-17.

Variables with slopes above the cut-off value(s) identified in any one or more of the sums shown in Table 7, Table 8 or Table 9 may be used as discriminators in the clustering algorithm.

In addition, although the examples given herein include those variables with positive correlation, those variables having negative correlation may also be taken into account. For example, reciprocal changes in positive and negative correlated variables may be expected. That is, for those biologic variables with negative correlation, the process may want to treat when they are at lower concentration, low abundance, or showing a declining trend, for example.

Time-dependent fluctuations' profiles of the selected immune variables are used to determine the optimum time of chemotherapy delivery by using the following method. Cosine profiles of the fluctuations may be clustered with the aim to find time window, during which the frequency of peak values of the index Π is the highest. The clustering is done by the K-means method with modifications. K-means clustering requires a priori knowledge of the number of clusters in which the objects (profiles) will be grouped. By this method, the number of groups is determined from the number of full function periods which fit into one observation period. The maximum possible number of groups equals the maximum number of function periods and the minimum number of groups equals the minimum number of function periods which fit into one observation period. The algorithm computes the number of clusters for the whole range of integers from the maximum to the minimum numbers. For each iteration (number of clusters) and for each variable the algorithm calculates the dates when the Π index has maximum value. These dates are used as centroids for K-means clustering. Since the result of K-means clustering depends on the order of initial centroids, the example modification performs clustering for all possible combinations of centroids and then computes the date when the sum of indices for all clustered cosine profiles was maximal. Next, the algorithm computes the dates with maximum sum of relative Π indices across all possible combination of centroids and all numbers of clusters. These dates are outputted as favorable dates for chemotherapy application for a given patient and a given set of immune variables (FIG. 2).

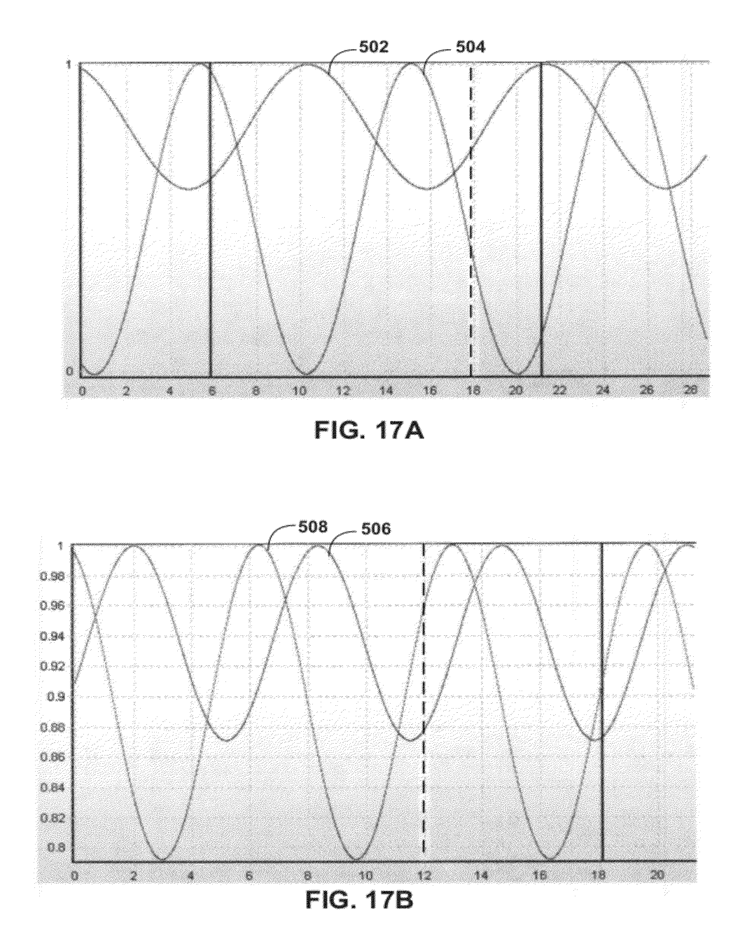

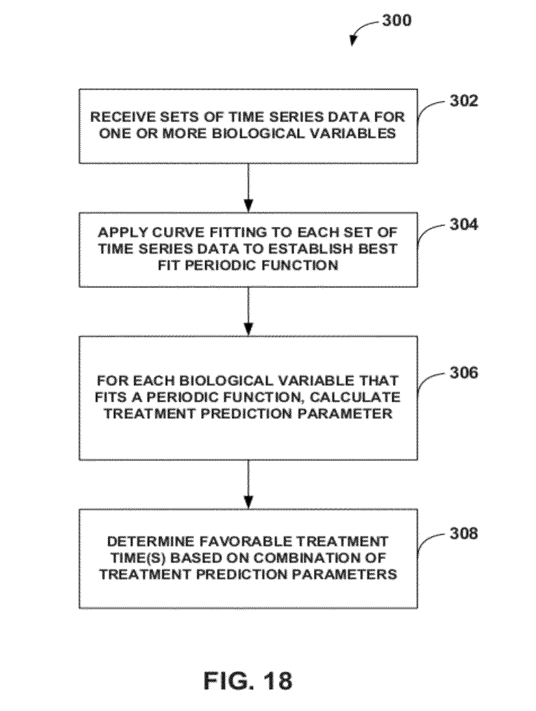

FIGS. 17A and 17B are graphs illustrating example clustering of concentration profiles IL-1ra (502) and IL-12p70 (504) in Patient #1 (PFS=916 days) (FIG. 17A) and concentration profiles IL-1ra (506) and IL-12p70 (508) in Patient #2 (PFS=37 days) (FIG. 17B). Black vertical lines represent dates, predicted by the clustering algorithm; dashed vertical lines represent dates when chemotherapy was started. In this example, three variables were clustered, but profiles for only two variables are shown on the plots for each patient. This resulted from filtering out profiles which did not satisfy the threshold criteria (in this case the goodness-of-fit criterion (R² value)) for a specific variable in an individual patient. The corresponding graph illustrating the association between the 5-day period of chemotherapy application, time predicted by the clustering algorithm and progression-free survival time in 8 melanoma patients is shown in FIG. 15.

Although in FIGS. 17A and 17B the variables used to determine treatment time(s) are the same (e.g., IL-1ra and IL-12p70) for each of the two patients, it shall be understood that this need not be the case. For example, the analysis may determine that for certain patients only one immune variable satisfies the threshold criteria, while for other patients two or more immune variables may satisfy the threshold criteria. In addition, the immune variables satisfying the threshold criteria may be different for different patients. The determination of favorable treatment times may therefore be patient-specific in the sense that only those biological variables satisfying desired threshold values may be used to determine favorable treatment times for each individual patient.

The example systems and/or methods described herein analyze time-dependent fluctuations of at least one biological variable measured in blood samples obtained from clinical patients and determine one or more favorable times for the pharmacological treatment of the patient. The systems and/or methods determine favorable time(s) for chemotherapy delivery based on serial measurements of one or more biological variables. In some examples, the biological variables are immune variables.

Each new series of experimental measurements may be processed according to the described workflow. This iterative computation of simulated parameters based on ever growing experimental evidence may iteratively enhance statistical power accuracy of p-values and overall precision in detecting functions to which the data fits. This, in turn, may enhance the accuracy of prediction of one or more favorable date(s) for chemotherapy treatment.

FIG. 18 is a flowchart illustrating an example process 300 by which a controller, such as controller 202 of system 200 shown in FIG. 10, may determine favorable treatment time(s) for delivery of chemotherapy treatment (or other type of pharmacological treatment) in a patient. The controller may receive sets of time series data for one or more biological variables (302). The biological variables may include, for example, immune variables. The immune variables may include, for example, IL-10, IL-12p(70), G-CSF, IL-9, VEGF, CD206, IL-1rα, IL-13, IL-15, IL-17, CD4/294, CD11c/14, CD197/CD206, and/or DR(hi). However, other immune or biological variables may also be included, and the disclosure is not limited in this respect.

The controller may apply curve fitting to each set of time series data to establish a best fit periodic function (304), if any. That is, the controller may determine whether each set of time series fits a periodic function. The controller may also determine the best-fit periodic function, if any, for each set of time series data. The periodic function may include, for example, a sinusoidal function, such as a sine or cosine function, any of the periodic functions described herein, or any other periodic function. For each biological variable that fits a periodic function, the controller may calculate a treatment prediction parameter (for example, the parameter or index Π as described herein) (306). The treatment prediction parameter may be based on, for example, the relative concentration of the biological variable and the relative derivative of the best fit periodic function. The controller may determine one or more relatively more favorable treatment time(s) based on a combination of the treatment prediction parameters (308). For example, the controller may sum or otherwise combine the treatment prediction parameters to arrive at a combined treatment prediction parameter.

The techniques described in this disclosure, including functions performed by a processor, controller, control unit, or control system, may be implemented within one or more of a general purpose microprocessor, digital signal processor (DSP), application specific integrated circuit (ASIC), field programmable gate array (FPGA), programmable logic devices (PLDs), or other equivalent logic devices. Accordingly, the terms “processor” “processing unit” or “controller,” as used herein, may refer to any one or more of the foregoing structures or any other structure suitable for implementation of the techniques described herein.

The various components illustrated herein may be realized by any suitable combination of hardware, firmware, and/or software. In the figures, various components are depicted as separate units or modules. However, all or several of the various components described with reference to these figures may be integrated into combined units or modules within common hardware, firmware, and/or software. Accordingly, the representation of features as components, units or modules is intended to highlight particular functional features for ease of illustration, and does not necessarily require realization of such features by separate hardware, firmware, or software components. In some cases, various units may be implemented as programmable processes performed by one or more processors or controllers.

Any features described herein as modules, devices, or components may be implemented together in an integrated logic device or separately as discrete but interoperable logic devices. In various aspects, such components may be formed at least in part as one or more integrated circuit devices, which may be referred to collectively as an integrated circuit device, such as an integrated circuit chip or chipset. Such circuitry may be provided in a single integrated circuit chip device or in multiple, interoperable integrated circuit chip devices, and may be used in any of a variety of applications and devices.

If implemented in part by software, the techniques may be realized at least in part by a computer-readable data storage medium comprising code with instructions that, when executed by one or more processors or controllers, performs one or more of the methods described in this disclosure. The computer-readable storage medium may form part of a computer program product, which may include packaging materials. The computer-readable medium may comprise random access memory (RAM) such as synchronous dynamic random access memory (SDRAM), read-only memory (ROM), non-volatile random access memory (NVRAM), electrically erasable programmable read-only memory (EEPROM), embedded dynamic random access memory (eDRAM), static random access memory (SRAM), flash memory, magnetic or optical data storage media. Any software that is utilized may be executed by one or more processors, such as one or more DSP's, general purpose microprocessors, ASIC's, FPGA's, or other equivalent integrated or discrete logic circuitry.

Various examples have been described. These and other examples are within the scope of the following claims.

1. A method comprising:
receiving time series data of immune variable concentration for an observed time period for each of a plurality of identified immune variables;
fitting a periodic function to the time series data corresponding to each of the plurality of identified immune variables;
calculating a treatment prediction parameter based on a relative concentration and a relative differential of the fitted periodic function for each time series data that fits a periodic function;
choosing the proposed treatment date such that the treatment prediction parameter is maximized; and
reporting the proposed date of treatment that maximizes the treatment prediction parameter.

2. The method of claim 1 further comprising:
determining a relative concentration of the fitted periodic function based on a maximum immune variable concentration within the observed time period, a minimum immune variable concentration within the observed time period, and an extrapolated immune variable concentration on a proposed treatment date;
determining a relative derivative of the fitted periodic function based on a maximum derivative within the observed time period, a minimum derivative within the same period, and an extrapolated derivative on the proposed treatment date;

3. The method of claim 1, wherein fitting a periodic function to the time series data comprises fitting each set of time series data to a cosine function.

4. The method of claim 1 wherein receiving time series data of immune variable concentration for an observed time period for each of a plurality of identified immune variables comprises receiving time series data of immune variable concentration for an observed time period for one or more of IL-10, IL-12p(70), G-CSF, IL-9, VEGF, CD206, IL-1rα, IL-13, IL-15, IL-17, CD4/294, CD11c/14, CD197/CD206, and DR(hi).

5. A computer-readable medium encoded with instructions that cause a programmable processor to:
receive time series data of immune variable concentration for an observed time period for each of a plurality of identified immune variables;
fit a periodic function to the time series data corresponding to each of the plurality of identified immune variables;
define a relative concentration of the fitted periodic function based on a maximum immune variable concentration within the observed time period, a minimum immune variable concentration within the observed time period, and an extrapolated immune variable concentration on a proposed treatment date;
define a relative derivative of the fitted periodic function based on a maximum derivative within the observed time period, a minimum derivative within the same period, and an extrapolated derivative on the proposed treatment date;
calculate a treatment prediction parameter based on the relative concentration and the relative differential;
choose the proposed treatment date such that the treatment prediction parameter is maximized; and
report the proposed date of treatment that maximizes the treatment prediction parameter.

6. A system comprising:
a controller that receives a set of time series data of immune variable concentration for an observed time period for each of a plurality of identified immune variables;
a curve-fitting module executed by the controller that fits a periodic function to the set of time series data corresponding to each of the plurality of identified immune variables;
a treatment prediction parameter module executed by the controller that calculates a treatment prediction parameter based on a relative concentration and a relative differential of the periodic function for each set of time series data that fits a periodic function;
a proposed treatment date module executed by the controller that chooses the proposed treatment date such that the treatment prediction parameter is maximized; and
a reporting module executed by the controller that generates a report concerning the proposed date of treatment that maximizes the treatment prediction parameter.

7. The system of claim 6, wherein the treatment prediction module further:
defines the relative concentration of the fitted periodic function based on a maximum immune variable concentration within the observed time period, a minimum immune variable concentration within the observed time period, and an extrapolated immune variable concentration on a proposed treatment date; and
defines the relative derivative of the fitted periodic function based on a maximum derivative within the observed time period, a minimum derivative within the same period, and an extrapolated derivative on the proposed treatment date.

8. A method comprising:
receiving a plurality of time series data of immune variable concentration in a patient for an observed time period each corresponding to a different one a plurality of identified immune variables;
determining for which of the identified immune variables the corresponding time series of immune variable concentration fit a periodic function;
for those immune variables that fit a periodic function, defining a relative concentration of the fitted periodic function based on a maximum immune variable concentration within the observed time period, a minimum immune variable concentration within the observed time period, and an extrapolated immune variable concentration on a proposed treatment date;
for those immune variables that fit a periodic function, defining a relative derivative of the fitted periodic function based on a maximum derivative within the observed time period, a minimum derivative within the same period, and an extrapolated derivative on the proposed treatment date;
calculating a treatment prediction parameter based on the relative concentration and the relative differential;
choosing the proposed treatment date such that the treatment prediction parameter is maximized; and
reporting the proposed date of treatment for the patient that maximizes the treatment prediction parameter.

9. The method of claim 8 wherein calculating a treatment prediction parameter based on the relative concentration and the relative differential comprises calculating a parameter Pi (Π) according to the equation:
Π=e^{der×T}×e^{conc}, where
der is the relative derivative,
conc is the relative concentration, and
T is a function period in days to correct for variable period length.

10. The method of claim 8 wherein calculating a treatment prediction parameter based on the relative concentration and the relative differential comprises calculating a parameter Pi (Π) according to the equation:
Π=(der×T)+conc, where
der is the relative derivative,
conc is the relative concentration, and
T is a function period in days to correct for variable period length.

11. The method of claim 8 wherein determining for which of the identified immune variables the corresponding time series of immune variable concentration fit a periodic function comprises determining which if the identified immune variables that corresponding time series fit a logistic function, a quadratic function, a cosine function, a rational function, a Gaussian function, or a Morgan-Mercer-Flodin (MMF) function.

12. A method of determining one or more favorable treatment times for delivery of pharmacological treatment to a patient, comprising:
receiving a set of time series data for each of one or more biological variables;
determining whether each set of time series data fit a periodic function;
for each biological variable that fits a periodic function, computing a treatment prediction parameter; and
determining one or more favorable treatment times based on the treatment prediction parameter calculated for at least one of the biological variables that fits a periodic function.

13. The method of claim 12 wherein receiving a set of time series data for each of one or more biological variables comprises receiving a set of time series data for one or more immune variables.

14. The method of claim 12 receiving a set of time series data for each of one or more biological variables comprises receiving a set of time series data for one or more of IL-10, IL-12p(70), G-CSF, IL-9, VEGF, CD206, IL-1rα, IL-13, IL-15, IL-17, CD4/294, CD11c/14, CD197/CD206, and DR(hi).

15. The method of claim 12 wherein determining whether each set of time series data fit a periodic function comprises determining whether each set of time series data fit a cosine function.

16. The method of claim 12 further comprising determining a best fit periodic function for each of the biological parameters.

17. The method according to claim 16, wherein computing a treatment prediction parameter comprises computing a treatment prediction parameter based on the relative concentration of the biological variable and the relative derivative of the best fit periodic function.

18. The method of claim 12 further comprising, for each biological variable that fits a periodic function, determining a relative concentration of the periodic function based on a maximum immune variable concentration within an observed time period, a minimum immune variable concentration within the observed time period, and an extrapolated immune variable concentration on a proposed treatment date.

19. The method of claim 12 further comprising, for each biological variable that fits a periodic function, determining a relative derivative of the periodic function based on a maximum derivative within an observed time period and a minimum derivative within the observed time period, and an extrapolated derivative on a proposed treatment date.
